(19) United States
(12) Patent Application Publication
(10) Pub. No.: US 2025/0074973 A1
(43) Pub. Date: Mar. 6, 2025

(54) COMPOSITIONS AND METHODS FOR TREATING HUNTINGTON’S DISEASE

(71) Applicant: Annexon, Inc., Brisbane, CA (US)

(72) Inventors: Ann Mongan, Brisbane, CA (US); Ted Yednock, Forest Knolls, CA (US); Ellen Cahir-McFarlaand, San Francisco, CA (US)

(21) Appl. No.: 18/725,580

(22) PCT Filed: Dec. 29, 2022
(86) PCT No.: PCT/US2022/082523

§ 371 Date: Jun. 28, 2024

Related U.S. Application Data
(60) Provisional application No. 63/349,305, filed on Jun. 6, 2022.
(60) Provisional application No. 63/295,213, filed on Dec. 30, 2021.

(51) Int. Cl. C07K 16/18; A61K 39/00; A61P 25/28

(52) U.S. Cl. CPC C07K 16/18; A61P 25/28; A61K 2039/505; A61K 2039/545; C07K 2317/76

(57) ABSTRACT

The present disclosure relates generally to methods of treating Huntington's disease in a subject in need thereof. The method comprises determining that the subject has an elevated level of C4a or an elevated C4a/C4 ratio; and administering to the subject an inhibitor of the classical complement pathway.

### RELATED APPLICATIONS

This patent application claims priority to U.S. Provisional Patent Application No. 63/349,305, filed Jun. 6, 2022, and U.S. Provisional Patent Application No. 63/295,213, filed Dec. 30, 2021, each of which is hereby incorporated by reference in its entirety.

### BACKGROUND

Huntington's Disease (HD) is an autosomal-dominant progressive neurodegenerative condition that is characterized by chorea and dystonia, cognitive decline and psychiatric disorder. The specific genetic mutation involves expansion of a trinucleotide (cytosine, adenine, and guanine [CAG]) repeat in the huntingtin gene (Htt) on chromosome 4p. The unstable CAG repeat is translated into a polyglutamine stretch in the huntingtin protein. The age of onset and severity of disease is found to be linked to the number of CAG repeats in the Htt gene with those individuals with the earliest onset having the largest repeat number. The normal function of the huntingtin protein is not known but current understanding supports the mutant protein with the expanded polyglutamine sequence as toxic to brain cells.

In general HD symptoms worsen progressively and invariably lead to death. HD is often divided into three broad phases. In early stage HD patients experience minor symptoms but are largely functional and able to work and live independently. In middle stage HD, patients' symptoms are more prominent including chorea and cognitive decline and they may not be able to work or care for themselves. In late stage HD, patients require assistance for all aspects of daily living, with dementia, mutism, dystonia, and bradykinesia predominating in advanced disease. Death typically occurs within 15-20 years of disease onset, and is usually related to complications of immobility, infection, especially pneumonia, and cardiac disease. The Unified Huntington's Disease Rating scale (UHDRS) first published in 1996 and revised in 1999 and 2005 is often used to stage patients with HD based on assessing motor function, cognition, behavior, independence, functional ability and a total functional capacity.

Neuropathologically, HD is primarily characterized by neuronal loss in the striatum and cerebral cortex. Certain neuronal populations are more affected including those within the corpus striatum of the basal ganglia. The mechanism by which polyglutamine aggregation leads to neurodegeneration has been elusive, although insight has emerged from animal models regarding HD pathophysiology. Early synaptic dysfunction is a key characteristic and is manifested by dysregulated glutamate release in striatum followed by progressive disconnection between cortex and striatum. Some of the alterations in late HD could be compensatory mechanisms designed to cope with early synaptic and receptor dysfunctions. These findings suggest that HD treatments need to be designed according to the stage of disease progression, however synaptic dysfunction and loss occur early and continue throughout disease progression.

To date, no treatment has been shown to delay the onset of HD or slow its progression. Treatment is focused on specific symptom management. A patient's care may include a broad range of physicians to address the various physical and psychological symptoms. Chorea is often linked to cognitive and psychological aspects of the disease such as anxiety and depression. Disabilities in one area usually lead to problems in another. Further, there is an unmet need to identify mediators of neuronal dysfunction in HD and to characterize natural history so that therapeutic effects can be accurately assessed. Therefore, there is a need in the art for new therapies to prevent and treat HD.

### SUMMARY

The present disclosure is generally directed to methods of treating Huntington's disease in a subject in need thereof. The method comprises determining that the subject has an elevated level of C4a or an elevated C4a/C4 ratio; and administering to the subject an inhibitor of the classical complement pathway, e.g., if the subject has an elevated level of C4a or an elevated C4a/C4 ratio. A therapeutically effective amount of the inhibitor may be administered.

The elevated level of C4a may be greater than a C4a level in normal or healthy subjects, such as subjects of a similar age. The elevated level of C4a may be greater than a reference C4a level. For example, the reference C4a level is a value that is equal to or greater than the median of C4a levels in samples derived from Huntington's disease subjects, such as subjects of a similar age. In other embodiments, the reference C4a level is a value that is equal to or greater than the 75th percentile of C4a levels in samples derived from normal or healthy subjects (subjects that do not have Huntington's disease.), such as subjects of a similar age.

In some embodiments, the elevated level of C4a is greater than the reference C4a level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%. In some embodiments, the elevated level of C4a is greater than the reference C4a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

The elevated C4a/C4 ratio may be greater than a C4a/C4 ratio in normal or healthy subjects, such as subjects of a similar age. In some embodiments, the elevated C4a/C4 ratio is greater than a reference C4a/C4 ratio. The reference C4a/C4 ratio may be a value that is equal to or greater than the median of C4a/C4 ratio in samples derived from Huntington's disease subjects, such as subjects of a similar age. In other embodiments, the reference C4a/C4 ratio is a value that is equal to or greater than the 75th percentile of C4a/C4 ratios in samples derived from normal or healthy subjects (subjects that do not have Huntington's disease), such as subjects of a similar age.

In some embodiments, the elevated C4a/C4 ratio is greater than the reference C4a/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%. In some embodiments, the elevated C4a/C4 ratio is greater than the reference C4a/C4 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the level of C4a or the C4a/C4 ratio is measured in cerebrospinal fluid (CSF) or plasma. In some embodiments, the subject has an elevated level of Neurofilament light chain (NfL). The elevated level of NfL may be greater than a NfL level in normal or healthy subjects, such as subjects of a similar age. In some embodiments, the elevated level of NfL is greater than a reference NfL level. The reference NfL level may be about 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1100 pg/ml, 1200 pg/ml, 1300 pg/ml, 1400 pg/ml, 1500 pg/ml, 1600 pg/ml, 1700 pg/ml, 1800 pg/ml, 1900 pg/ml, or 2000 pg/ml. In some embodiments, the elevated level of NfL is greater than the NfL level in normal or healthy subjects or the reference NfL level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, or 5000%. In some embodiments, the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%. In some embodiments, the level of NfL is measured in cerebrospinal fluid (CSF).

In other embodiments, the level of NfL is measured in plasma. The reference NfL level may be about 1 pg/ml, 2 pg/ml, 3 pg/ml, 4 pg/ml, 5 pg/ml, 6 pg/ml, 7 pg/ml, 8 pg/ml, 9 pg/ml, 10 pg/ml, 11 pg/ml, 12 pg/ml, 13 pg/ml, 14 pg/ml, 15 pg/ml, 16 pg/ml, 17 pg/ml, 18 pg/ml, 19 pg/ml, or 20 pg/ml. In some embodiments, the elevated level of NfL is greater than the NfL level in normal or healthy subjects or the reference NfL level by at least 1%, 2%, 3%, 4%, 5%, 6%, 7%, 8%, 9%, 1%, %15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, or 5000%. In some embodiments, the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%.

In some embodiments, the inhibitor of the classical complement pathway is a C1q inhibitor, such as a small molecule, an antibody, an aptamer, an antisense nucleic acid or a gene-editing agent. The antibody may be an anti-C1q antibody. The antibody may be administered at a dose of at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg. In some embodiments, the antibody is administered at a dose of 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 120 mg/kg, 130 mg/kg, 140 mg/kg, or 150 mg/kg. In some embodiments, the antibody is administered at a dose of 75 mg/kg on day 1 and on day 5 or day 6. In some embodiments, the antibody is further administered at a dose of 100 mg/kg every two weeks.

In some embodiments, the antibody is administered intravenously. In some embodiments, the antibody is administered once a week, once every other week, once a month, once every six weeks, or once every other month. In some embodiments, the antibody is administered for at least 3 months, at least 6 months, at least 7 months, at least 8 months, at least 9 months, at least 10 months, at least 11 months, or at least 12 months. In some embodiments, the antibody is administered for 3 months, 6 months, 7 months, 8 months, 9 months, 10 months, 11 months, or 12 months.

In some embodiments, the anti-C1q antibody inhibits the interaction between C1q and an autoantibody or between C1q and C1r, or between C1q and C1s, or the anti-C1q antibody promotes clearance of C1q from circulation or a tissue. In some embodiments, the antibody is a monoclonal antibody, a polyclonal antibody, a recombinant antibody, a humanized antibody, a chimeric antibody, a multispecific antibody, antibody fragments, or an antibody derivative thereof. The antibody fragment may be a Fab fragment, a Fab′ fragment, a F(ab′)2 fragment, a Fv fragment, a diabody, a single chain antibody molecule, or a single arm antibody molecule.

In some embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7, and/or a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7, preferably the light chain variable domain comprising an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11, preferably the heavy chain variable domain comprising an amino acid sequence selected from SEQ ID NO: 8 and 31-34. In some embodiments, the antibody fragment comprises heavy chain Fab fragment of SEQ ID NO: 39 and light chain Fab fragment of SEQ ID NO: 40.

In some embodiments, the inhibitor of the classical complement pathway is a C1r inhibitor, such as a small molecule, an antibody, an aptamer, an antisense nucleic acid or a gene editing agent, preferably an anti-C1r antibody. In some embodiments, the anti-C1r antibody inhibits the interaction between C1r and C1q or between C1r and C1s, or wherein the anti-C1r antibody inhibits the catalytic activity of C1r or inhibits the processing of pro-C1r to an active protease.

In some embodiments, the inhibitor of the classical complement pathway is a C1s inhibitor, such as a small molecule, an antibody, an aptamer, an antisense nucleic acid or a gene editing agent, preferably an anti-C1s antibody. In some embodiments, the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s. In some embodiments, the antibody is sutimlimab.

In some embodiments, the inhibitor of the classical complement pathway is an anti-C1 complex antibody, optionally wherein the anti-C1 complex antibody inhibits C1r or C1s activation or blocks their ability to act on C2 or C4. The anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s.

In some embodiments, the inhibitor of the classical complement pathway is a C2 inhibitor, such as a small molecule, an antibody, an aptamer, an antisense nucleic acid or a gene editing agent.

In some embodiments, the inhibitor of the classical complement pathway is a C3 inhibitor, such as a small molecule, an antibody, an aptamer, an antisense nucleic acid or a gene editing agent. The C3 inhibitor may be APL-9 (Apellis) or AMY-101 (Amyndas).

In some embodiments, the inhibitor of the classical complement pathway is a C4 inhibitor, such as a small molecule, an antibody, an aptamer, an antisense nucleic acid or a gene editing agent.

In one aspect, disclosed here is a method of treating Huntington's disease in a subject in need thereof. The method comprises determining that the subject has an elevated level of C4a or an elevated C4a/C4 ratio. The method may further comprise administering to the subject an antibody having a light variable domain comprising an amino acid sequence of SEQ ID NO: 37 and a heavy chain variable domain comprising the amino acid sequence of SEQ ID NO: 33. In some embodiments, the antibody is administered intravenously at a dose of at least 75 mg/Kg on day 1 and day 5 or day 6. The antibody may be further administered intravenously at a dose of 100 mg/Kg every two weeks. In some embodiments, the antibody comprises a heavy chain comprising an amino acid sequence of SEQ ID NO: 14 and a light chain comprising an amino acid sequence of SEQ ID NO: 40.

### DESCRIPTION OF THE FIGURES

FIGS. 1A-1B show that inhibition of C1q blocks classical complement activation and provides synapse protection in a preclinical animal model of HD. FIG. 1A shows that inhibition of excess complement blocks deposition on synapses (Fold-Change in Complement Deposition on Synapses). FIG. 1B shows that inhibition of excess complement protects against synapse loss (Fold-Change Synapse Number).

FIG. 2 depicts cerebrospinal fluid (“CSF”) C4a elevated and increasing with HD progression. The data analyzed is from the natural history study.

FIG. 3 shows that C4a increases in the CSF of manifest HD patients. The data analyzed is from clinical study in Example 2.

FIGS. 4A-4B show that neurofilament light (“NFL” or “Nfl”) levels are significantly elevated in CSF of manifest HD patients and CSF NFL correlates with CSF C4a (HDClarity study). FIG. 4A shows elevated NFL in CSF of manifest HD patients. FIG. 4B shows that CSF NFL correlates with CSF C4a.

FIG. 5 shows that C4a can inform disease stage beyond predicted by age. Errors are residuals or mis-category made by the models. Each data point corresponds to 1 patient. Model LM1: HD disease stage predicted by age. Model LM2: HD disease stage predicted by age+C4a. LM2, which includes C4a in addition to age, predicts HD disease stage more accurately compared to LM1. The diagonal line represents equal accuracy between two models. Data below the diagonal line indicates that LM2 has lower error, hence higher accuracy, than LM1

FIGS. 6A-6B show that model combining complement activation, NfL and Age more accurately predicts HD stage than NfL alone. FIG. 6A shows model HD progression predicted by Nfl alone. FIG. 6B shows model HD stage predicted by Nfl, age, and complement.

FIGS. 7A-7B show approximately 50% of HD patients have elevated CSF levels of complement activation product C4a. FIG. 7A shows elevated C4a in CSF of natural history patients. FIG. 7B shows similar range of C4a in current study cohort.

FIG. 8 shows CSF C4a/C4 ratio: a sensitive measure of ongoing complement activation. C4 decreased (consumed) with C1q activation activity. C4a increased (produced) with C1q activation. C4a/C4 ratio corrects for genetic variability among subjects.

FIG. 9 shows analyzed HD phase 2 data using CSF C4a/C4 ratio as measure of ongoing complement activity. Patients divided into two groups based on C4a/C4 levels at baseline. Characteristics of two groups balanced at baseline.

FIG. 10 shows that high baseline complement patients showed consistent and significant improvement on cUHDRS over 24-week treatment period.

FIG. 11A-11D show high baseline C4a/C4 ratio patients consistently improved on cUHDRS sub-domains over 24-week treatment period. FIG. 11A shows Symbol Digit Modality Test (Cognition). FIG. 11B shows total functional capacity (TFC, activities of daily living). FIG. 11C shows stroop word reading test (SWR, Cognition). FIG. 11D shows total motor score (TMS).

FIGS. 12A-12B show that 75% of patients with high baseline complement activity (C4a/C4) improved at week 24 in cUHDRS vs. 36% with low activation. Biomarker-differentiated responses unlikely to be placebo driven. FIG. 12A shows that 13/23 (56%) of all patients improved at Week 24. FIG. 12B shows that 9/12 (75%) patients improved with high baseline complement activation while 4/11 (36%) improved with low baseline complement activation.

FIG. 13 shows that plasma NfL levels are consistent with HD natural history at week 24.

FIG. 14 shows that CSF NfL levels are consistent with HD natural history at week 24.

FIGS. 15A-15B show combined baseline C4a ratio, NfL and age identifies separate groups of differential cUHDRS at week 24. FIG. 15A shows model prediction compared to observed cUHDRS. FIG. 15B shows patient performance by groups identified by model. Improvers are significantly higher than non-improvers. P-val=0.009. Wilcoxon, 1-tail test.

FIGS. 16A-16B show that full-length C1q antibody demonstrated rapid, robust and long-lasting complement engagement. FIG. 16A shows full-length C1q antibody levels in CSF. Note: full-length C1q antibody concentrations <LLOQ are shown as 0.1 μg/mL so they show up on log scale. FIG. 16B shows full engagement of C1q in CSF. Note: C1q concentrations <LLOQ are shown as 0.25 μg/mL so they show up on log scale. *Data from discontinued subjects were included until dosing was discontinued.

FIGS. 17A-17B show that full-length C1q antibody demonstrated rapid, robust and long-lasting complement engagement. FIG. 17A shows full-length C1q antibody levels in serum. FIG. 17B shows full engagement of C1q in serum. Note: C1q concentrations <LLOQ are shown as 0.25 μg/mL so they show up on log scale. *Data from discontinued subjects were included until dosing was discontinued.

FIG. 18 shows prolonged full-length C1q antibody effect throughout 3-month off-treatment period. C4a release is a pharmacodynamic marker of C1q target engagement.

FIGS. 19A-19B show full-length C1q antibody treatment resulted in early and sustained improvement in patients with excess baseline complement activity (Comparing higher vs lower C4a/C4 groups at week 24). FIG. 19A shows Unified Huntington's Disease Rating Scale (cUHDRS). FIG. 19B shows total functional capacity (TFC, activities of daily living). * MMRM; LS means+/−95% CI. Interpolated natural history from Schobel 2017 (TRACK-HD).

FIGS. 20A-20D show that benefit in patients with high complement activity was demonstrated across most cUHDRS constituent domains. FIG. 20A shows total functional capacity (TFC, activities of daily living). FIG. 20B shows Symbol Digit Modality Test (SDMT, Cognition). FIG. 20C shows total motor score (TMS). FIG. 20D shows stroop word reading test (SWR, Cognition). *MMRM; LS means+/−95% CI; Comparing higher vs. lower groups at week 24. N=23.

FIG. 21 shows that plasma NfL levels are consistent with HD natural history at week 36 with 4% change from screening.

FIG. 22 shows that CSF NfL levels are consistent with HD natural history at week 36 with 16% change from screening. *Results are independent of baseline complement activity.

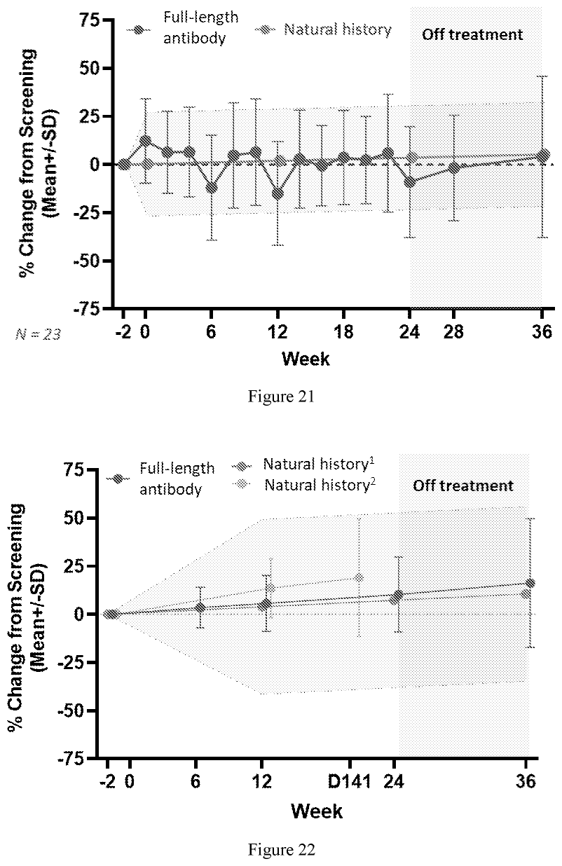

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

Complement activation has been implicated in the aberrant removal of synapses during neurodegenerative disease, and in conjunction with its associated inflammation may contribute to ongoing neuronal loss. Complement expression and activation are elevated in many neurodegenerative diseases, including HD, Amyotrophic Lateral Sclerosis (ALS) and Alzheimer's disease (AD) patients. Therapies targeting the complement pathway may inhibit the complement driven functional synapse loss and neuronal damage in HD and reduce disease progression.

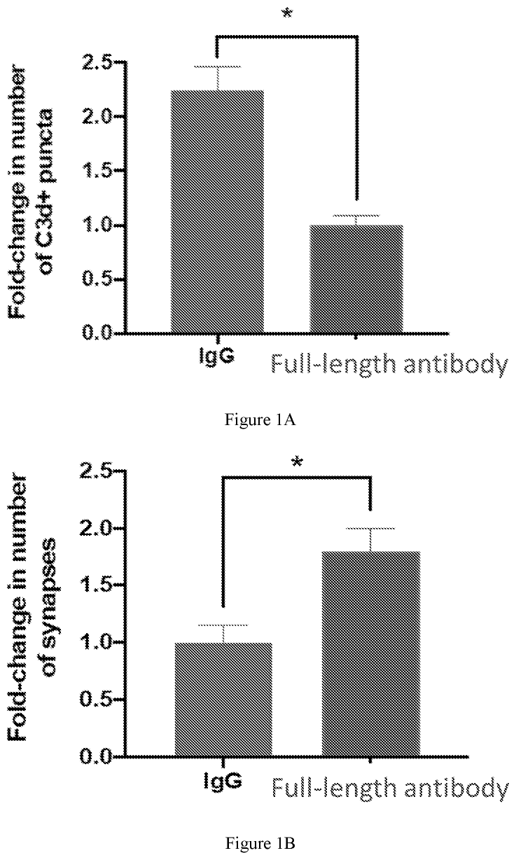

In HD and other neurodegenerative diseases, synaptic dysfunction and elimination occurs prior to axonal and neuronal loss, which are associated with NfL release. Complement activation and synaptic dysfunction and elimination correlates with early and progressive functional decline throughout disease process. Inhibition of C1q blocks classical complement activation and provides synapse protection in a preclinical model of HD. Inhibition of excess complement blocks deposition on synapses (FIG. 1A). Inhibition of excess complement protects against synapse loss (FIG. 1B).

The present disclosure presents a model, evaluating complement activation, alone or in combination with NfL and age, more accurately predicts HD stage than NfL alone or in combination with age. Data from the HD clinical trial (in Example 2) demonstrate that patients identified via the use of a model evaluating high C4a levels and/or high C4a/C4 ratios exhibit clinical improvement with anti-C1q antibody treatment. The clinical trial data also demonstrate treatment with full-length anti-C1q antibody leads to clinical benefits in HD patients identified by the use of a model evaluating complement activation alone or in combination with NfL and age. The clinical trial data also demonstrate that accounting for complement activation, NfL, and age, and the use of a full-length anti-C1q antibody reliably leads to clinical benefits in HD patients. Clinical benefits were measured by composite Unified Huntington's Disease Rating Scale (cUHDRS), or any of the following domains: cognitive (SDMT, SWR), total functional capacity (TFC), and total motor skills (TMS) (Kieburtz K., Mov Disord., 1996 March; 11(2):136-42 and Schobel S. A., et al., Neurology. 2017 Dec. 12; 89(24): 2495-2502).

The present disclosure is generally directed to methods of treating Huntington's disease in a subject in need thereof. The method comprises determining that the subject has an elevated level of C4a and/or an elevated C4a/C4 ratio; and administering to the subject so identified an inhibitor of the classical complement pathway.

All sequences mentioned in the present disclosure are incorporated by reference from U.S. Pat. No. 10,316,081, U.S. patent application Ser. No. 14/890,811, U.S. Pat. Nos. 8,877,197, 9,708,394, U.S. patent application Ser. No. 15/360,549, U.S. Pat. Nos. 9,562,106, 10,450,382, 10,457,745, International Patent Application No. PCT/US2018/022462 each of which is hereby incorporated by reference for the antibodies and related compositions disclosed.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

As used herein “reference level” relates to a predetermined criteria used as a reference for evaluating the values or data obtained from a sample obtained from an individual. The reference level can be an absolute value; a relative value; a value that has an upper or a lower limit; a range of values; an average value; a median value; a mean value; or a value as compared to a particular control or baseline value. A reference level can be based on an individual sample value, such as for example, a value obtained from a sample from the subject being tested, but at an earlier point in time. The reference level can be based on a large number of samples, such as from a population of subjects of similar chronological age, gender, disease state, or otherwise matched group, or based on a pool of samples including or excluding the sample to be tested. A reference level can also be determined from a representative number of samples (e.g., plasma or CSF) derived from different individuals afflicted with HD. A reference level can also be determined from biological samples from non-HD afflicted individuals (i.e., normal or healthy subjects of a similar age). These biological samples from a HD afflicted or non-HD afflicted individual may comprise for example, tissue biopsies, blood, plasma, serum, fecal samples, urine, cerebral spinal fluid, pap smears, or semen. A representative sample can include measurements from at least 10, 20, 30, 40, 50, 100, 200, 300, 400, 500, 600, 700, 800, 900, 1,000 or more individuals.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit the desired biological activity, and antibody derivatives.

The basic 4-chain antibody unit is a heterotetrameric glycoprotein composed of two identical light (L) chains and two identical heavy (H) chains. The pairing of a V_{H} and VL together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

The term “agent” as used herein describes any molecule, e.g. protein or pharmaceutical, with the capability of modulating synapse loss, particularly through the complement pathway. Candidate agents also include genetic elements, e.g., anti-sense and RNAi molecules to inhibit C1q expression, and constructs encoding complement inhibitors, e.g., CD 59, and the like. Candidate agents encompass numerous chemical classes, though typically they are organic molecules, including small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Generally, a plurality of assay mixtures are run in parallel with different agent concentrations to obtain a differential response to the various concentrations. Typically one of these concentrations serves as a negative control, i.e., at zero concentration or below the level of detection.

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous since they are typically synthesized by hybridoma culture, uncontaminated by other immunoglobulins. The modifier “monoclonal” indicates the character of the antibody as being obtained as a substantially homogeneous population of antibodies, and is not to be construed as requiring production of the antibody by any particular method. For example, the monoclonal antibodies to be used in accordance with the present disclosure may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

“Full-length antibodies” are usually heterotetrameric glycoproteins of about 150,000 daltons, comprising two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The terms “full-length antibody,” “intact antibody” and “whole antibody” are used interchangeably to refer to an antibody in its substantially intact form, as opposed to an antibody fragment or antibody derivative. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8(10):1057-1062 (1995)). Additional examples of antibody fragments include antibody derivatives such as single-chain antibody molecules, single-arm antibodies, antibodies with a single antigen-binding arm, monovalent antibodies and multispecific antibodies formed from antibody fragments

The term “single-arm antibody” herein is used to cover an antibody that comprises a single antigen-binding arm. The single-arm antibody may comprise an antigen-binding arm and an Fc region, wherein the single antigen-binding arm comprises a light chain variable domain and a heavy chain variable domain; and the Fc region comprises a complex of a first and a second Fc polypeptide. In some embodiments, one but not both of the Fc polypeptides is an N-terminally truncated heavy chain. In some embodiments, the antibody may be a bivalent antibody—where one arm binds C1q and the other binds a different antigen. Depending on the other antigen, such an antibody would not crosslink and activate C1q.

“An antibody with a single antigen-binding arm” as used herein means an antibody comprising a single antigen-binding arm and an Fc region, wherein the antigen-binding arm comprises a light chain variable domain and a heavy chain variable domain. In some embodiments, the antibody further comprises an inactive antigen-binding arm, which is incapable of binding to the antigen, or comprises an arm that binds to a different antigen. In some embodiments, the Fc region comprises a complex of a first and a second Fc polypeptide.

An “antibody derivative” is any construct that comprises the antigen binding region of an antibody. Examples of antibody derivatives include single-chain antibody molecules, single arm antibodies, antibodies with a single antigen-binding arm, monovalent antibodies and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (See, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

“Fv” is the minimum antibody fragment, which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 1993/011161; WO/2009/121948; WO/2014/191493; Hollinger et al., Proc. Nat'l Acad. Sci. USA 90:6444-48 (1993).

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology.

The term “hypervariable region,” “HVR,” or “HV,” when used herein refers to the regions of an antibody-variable domain that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, NJ, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

“Framework” or “FR” residues are those variable-domain residues other than the HVR residues as herein defined.

The phrase “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In some embodiments, an affinity-matured antibody has nanomolar or even picomolar affinities for the target antigen. Affinity-matured antibodies are produced by procedures known in the art. For example, Marks et al., Bio Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷M⁻¹, about 10¹ M⁻¹ to 10⁹M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between a complement protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

A “blocking” antibody, an “antagonist” antibody, an “inhibitory” antibody, or a “neutralizing” antibody is an antibody that inhibits or reduces one or more biological activities of the antigen it binds, such as interactions with one or more proteins. In some embodiments, blocking antibodies, antagonist antibodies, inhibitory antibodies, or “neutralizing” antibodies substantially or completely inhibit one or more biological activities or interactions of the antigen.

The term “inhibitor” refers to a compound having the ability to inhibit a biological function of a target biomolecule, for example, an mRNA or a protein, whether by decreasing the activity or expression of the target biomolecule. An inhibitor may be an antibody, a small molecule, or a nucleic acid molecule. The term “antagonist” refers to a compound that binds to a receptor, and blocks or dampens the receptor's biological response. The term “inhibitor” may also refer to an “antagonist.”

Antibody “effector functions” refer to those biological activities attributable to the Fc region (a native sequence Fc region or amino acid sequence variant Fc region) of an antibody, and vary with the antibody isotype.

As used herein, the term “affinity” refers to the equilibrium constant for the reversible binding of two agents (e.g., an antibody and an antigen) and is expressed as a dissociation constant (KD). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. For example, a subject anti-C1s antibody binds specifically to an epitope within a complement C1s protein. “Specific binding” refers to binding with an affinity of at least about 10⁻⁷ M or greater, e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

The term “kₒₙ”, as used herein, is intended to refer to the rate constant for association of an antibody to an antigen.

The term “k_{off}”, as used herein, is intended to refer to the rate constant for dissociation of an antibody from the antibody/antigen complex.

The term “K_{D}”, as used herein, is intended to refer to the equilibrium dissociation constant of an antibody-antigen interaction.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

“Chronic” administration refers to administration of the medicament(s) in a continuous as opposed to acute mode, so as to maintain the initial therapeutic effect (activity) for an extended period of time. “Intermittent” administration refers to treatment that is not administered consecutively without interruption, but rather is cyclic/periodic in nature.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### C1q Inhibitors

The inhibitor of the classical complement pathway may be a C1q inhibitor, such as a small molecule, an antibody, an aptamer, an antisense nucleic acid or a gene editing agent.

The anti-C1q antibodies disclosed herein are potent inhibitors of C1q.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂).

Suitable inhibitors include an antibody that binds complement factor C1q and/or C1q in the C1 complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:
C1q, chain A (Homo sapiens), Accession No. Protein
Data Base: NP 057075.1; GenBank No.: NM 015991:
>gi|7705753|ref|NP_057075.1|complement C1q
subcomponent subunit A precursor [Homo sapiens]

(SEQ ID NO: 1)
MEGPRGWLVLCVLAISLASMVTEDLCRAPDGKKGEAGRPGRRGRPGLKG
EQGEPGAPGIRTGIQGLKGDQGEPGPSGNPGKVGYPGPSGPLGARGIPG
IKGTKGSPGNIKDQPRPAFSAIRRNPPMGGNVVIFDTVITNQEEPYQNH
SGRFVCTVPGYYYFTFQVLSQWEICLSIVSSSRGQVRRSLGFCDTTNKG
LFQVVSGGMVLQLQQGDQVWVEKDPKKGHIYQGSEADSVFSGFLIFPSA.

C1q, chain B (Homo sapiens), Accession No. Protein
Data Base: NP_000482.3; GenBank No.: NM_000491.3:
>gi|87298828|ref|NP_000482.3|complement C1q
subcomponent subunit B precursor [Homo sapiens]

(SEQ ID NO: 41)
MMMKIPWGSIPVLMLLLLLGLIDISQAQLSCTGPPAIPGIPGIPGTPGP
DGQPGTPGIKGEKGLPGLAGDHGEFGEKGDPGIPGNPGKVGPKGPMGPK
GGPGAPGAPGPKGESGDYKATQKIAFSATRTINVPLRRDQTIRFDHVIT
NMNNNYEPRSGKFTCKVPGLYYFTYHASSRGNLCVNLMRGRERAQKVVT
FCDYAYNTFQVTTGGMVLKLEQGENVFLQATDKNSLLGMEGANSIFSGF
LLFPDMEA.

C1q, chain C (Homo sapiens), Accession No. Protein
Data Base: NP_001107573.1; GenBank No.:
NM_001114101.1:
>gi|166235903|ref|NP_001107573.1|complement C1q
subcomponent subunit C precursor [Homo sapiens]

(SEQ ID NO: 42)
MDVGPSSLPHLGLKLLLLLLLLPLRGQANTGCYGIPGMPGLPGAPGKDG
YDGLPGPKGEPGIPAIPGIRGPKGQKGEPGLPGHPGKNGPMGPPGMPGV
PGPMGIPGEPGEEGRYKQKFQSVFTVTRQTHQPPAPNSLIRFNAVLTNP
QGDYDTSTGKFTCKVPGLYYFVYHASHTANLCVLLYRSGVKVVTFCGHT
SKTNQVNSGGVLLRLQVGEEVWLAVNDYYDMVGIQGSDSVFSGFLLFPD.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, or a chimeric antibody.

Other anti-C1q antibodies suitable for binding to C1q protein are well-known in the art and include, for example, antibodies Cat #: AF2379, AF1696, MAB1696, and MAB23791 (R&D System), NBP1-87492, NB100-64420, H00000712-B01P, H00000712-D01P, and H00000712-D01 (Novus Biologicals), MA1-83963, MA1-40311, PA5-14208, PA5-29586, and PA1-36177 (ThermoFisher Scientific), ab71940, ab11861, ab4223, ab72355, ab182451, ab46191, ab227072, ab182940, ab216979, and ab235454 (abcam), etc. Moreover, multiple siRNA, shRNA, CRISPR constructs for reducing C1q expression can be found in the commercial product lists of the above-referenced companies, such as SiRNA product #sc-43651, sc-44962, sc-105153, sc-141842, ShRNA product #sc-43651-SH, sc-43651-V, sc-44962-SH, sc-44962-V, sc-105153-SH, sc-105153-V, sc-141842-SH, sc-141842-V, CRISPR product #sc-419385, sc-419385-HDR, sc-419385-NIC, sc-419385-NIC-2, sc-402156, sc-402156-KO-2, sc-404309, sc-404309-HDR, sc-404309-NIC, sc-404309-NIC-2, sc-419386, sc-419386-HDR, sc-419386-NIC, sc-419386-NIC-2 (Santa Cruz Biotechnology, etc).

Light Chain and Heavy Chain Hypervariable Region Sequences and Variable Domain Sequences of Antibody M1 (Incorporated by Reference from U.S. Pat. No. 9,708,394)

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWYQEKPGKTNKLLIY
SGSTLQSGIPSRFSGSGSGTDFTLTISSLEPEDFAMYYCQQHNEYPLTF
GAGTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the M1 light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHWVKQRPGQGLEWIG
VIHPNSGSINYNEKFESKATLTVDKSSSTAYMQLSSLTSEDSAVYYCAG
ERDSTEVLPMDYWGQGTSVTVSS

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIIIPNSGSINYNEKFES (SEQ ID NO:10), and the HVR-H3 of the M1 heavy chain variable domain has the sequence ERDSTEVLPMDY (SEQ ID NO:11).

The nucleic acid sequence encoding the light chain variable domain was determined to be:

(SEQ ID NO: 12)
GATGTCCAGATAACCCAGTCTCCATCTTATCTTGCTGCATCTCCTGGAG
AAACCATTACTATTAATTGCAGGGCAAGTAAGAGCATTAACAAATATTT
AGCCTGGTATCAAGAGAAACCTGGGAAAACTAATAAGCTTCTTATCTAC
TCTGGATCCACTTTGCAATCTGGAATTCCATCAAGGTTCAGTGGCAGTG
GATCTGGTACAGATTTCACTCTCACCATCAGTAGCCTGGAGCCTGAAGA
TTTTGCAATGTATTACTGTCAACAACATAATGAATACCCGCTCACGTTC
GGTGCTGGGACCAAGCTGGAGCTGAAA.

The nucleic acid sequence encoding the heavy chain variable domain was determined to be:

(SEQ ID NO: 13)
CAGGTCCAACTGCAGCAGCCTGGGGCTGAGCTGGTAAAGCCTGGGGCTT
CAGTGAAGTTGTCCTGCAAGTCTTCTGGCTACCATTTCACCAGCTACTG
GATGCACTGGGTGAAGCAGAGGCCTGGACAAGGCCTTGAGTGGATTGGA
GTGATTCATCCTAATAGTGGTAGTATTAACTACAATGAGAAGTTCGAGA
GCAAGGCCACACTGACTGTAGACAAATCCTCCAGCACAGCCTACATGCA
ACTCAGCAGCCTGACATCTGAGGACTCGGCGGTCTATTATTGTGCAGGA
GAGAGAGATTCTACGGAGGTTCTCCCTATGGACTACTGGGGTCAAGGAA
CCTCAGTCACCGTCTCCTCA.

### Deposit of Material

The following materials have been deposited according to the Budapest Treaty in the American Type Culture Collection, ATCC Patent Depository, 10801 University Blvd., Manassas, Va. 20110-2209, USA (ATCC):

| | | | ATCC |
|---|---|---|---|
| | | Deposit | Accession |
| Sample ID | Isotype | Date | No. |
| Mouse hybridoma C1qM1 | IgG1, | Jun. 6, | PTA-120399 |
| 7788-1(M) 051613 | kappa | 2013 | |
| producing | | | |
| anti-C1q antibody M1 | | | |

The hybridoma cell line producing the M1 antibody (mouse hybridoma C1qM1 7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

Disclosed herein are methods of administering an anti-C1q antibody comprising a light chain variable domain and a heavy chain variable domain. The antibody may bind to at least human C1q, mouse C1q, or rat C1q. The antibody may be a humanized antibody, a chimeric antibody, or a human antibody. The antibody may be a monoclonal antibody, an antibody fragment thereof, and/or an antibody derivative thereof. The light chain variable domain comprises the HVR-L1, HVR-L2, and HVR-L3 of the monoclonal antibody M1 produced by a hybridoma cell line deposited with Accession Number PTA-120399. The heavy chain variable domain comprises the HVR-H1, HVR-H2, and HVR-H3 of the monoclonal antibody M1 produced by a hybridoma cell line deposited with ATCC Accession Number PTA-120399.

In some embodiments, the amino acid sequence of the light chain variable domain and heavy chain variable domain comprise one or more of SEQ ID NO:5 of HVR-L1, SEQ ID NO:6 of HVR-L2, SEQ ID NO:7 of HVR-L3, SEQ ID NO:9 of HVR-H1, SEQ ID NO:10 of HVR-H2, and SEQ ID NO:11 of HVR-H3.

The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

Disclosed herein are methods of administering an anti-C1q antibody, which inhibits the interaction between C1q and an autoantibody. In preferred embodiments, the anti-C1q antibody causes clearance of C1q from the circulation or tissue.

In some embodiments, the anti-C1q antibody of this disclosure inhibits the interaction between C1q and C1s. In some embodiments, the anti-C1q antibody inhibits the interaction between C1q and C1r. In some embodiments, the anti-C1q antibody inhibits the interaction between C1q and C1s and between C1q and C1r. In some embodiments, the anti-C1q antibody inhibits the interaction between C1q and another antibody, such as an autoantibody. In preferred embodiments, the anti-C1q antibody causes clearance of C1q from the circulation or tissue. In some embodiments, the anti-C1q antibody inhibits the respective interactions, at a stoichiometry of less than 2.5:1; 2.0:1; 1.5:1; or 1.0:1. In some embodiments, the C1q antibody inhibits an interaction, such as the C1q-C1s interaction, at approximately equimolar concentrations of C1q and the anti-C1q antibody. In other embodiments, the anti-C1q antibody binds to C1q with a stoichiometry of less than 20:1; less than 19.5:1; less than 19:1; less than 18.5:1; less than 18:1; less than 17.5:1; less than 17:1; less than 16.5:1; less than 16:1; less than 15.5:1; less than 15:1; less than 14.5:1; less than 14:1; less than 13.5:1; less than 13:1; less than 12.5:1; less than 12:1; less than 11.5:1; less than 11:1; less than 10.5:1; less than 10:1; less than 9.5:1; less than 9:1; less than 8.5:1; less than 8:1; less than 7.5:1; less than 7:1; less than 6.5:1; less than 6:1; less than 5.5:1; less than 5:1; less than 4.5:1; less than 4:1; less than 3.5:1; less than 3:1; less than 2.5:1; less than 2.0:1; less than 1.5:1; or less than 1.0:1. In certain embodiments, the anti-C1q antibody binds C1q with a binding stoichiometry that ranges from 20:1 to 1.0:1 or less than 1.0:1. In certain embodiments, the anti-C1q antibody binds C1q with a binding stoichiometry that ranges from 6:1 to 1.0:1 or less than 1.0:1. In certain embodiments, the anti-C1q antibody binds C1q with a binding stoichiometry that ranges from 2.5:1 to 1.0:1 or less than 1.0:1. In some embodiments, the anti-C1q antibody inhibits the interaction between C1q and C1r, or between C1q and C1s, or between C1q and both C1r and C1s. In some embodiments, the anti-C1q antibody inhibits the interaction between C1q and C1r, between C1q and C1s, and/or between C1q and both C1r and C1s. In some embodiments, the anti-C1q antibody binds to the C1q A-chain. In other embodiments, the anti-C1q antibody binds to the C1q B-chain. In other embodiments, the anti-C1q antibody binds to the C1q C-chain. In some embodiments, the anti-C1q antibody binds to the C1q A-chain, the C1q B-chain and/or the C1q C-chain. In some embodiments, the anti-C1q antibody binds to the globular domain of the C1q A-chain, B-chain, and/or C-chain. In other embodiments, the anti-C1q antibody binds to the collagen-like domain of the C1q A-chain, the C1q B-chain, and/or the C1q C-chain.

Where antibodies of this disclosure inhibit the interaction between two or more complement factors, such as the interaction of C1q and C1s, or the interaction between C1q and C1r, the interaction occurring in the presence of the antibody may be reduced by at least 10%, at least 20%, at least 30%, at least 40%, at least 50%, at least 60%, at least 70%, at least 80%, at least 90%, at least 95%, or at least 99% relative to a control wherein the antibodies of this disclosure are absent. In certain embodiments, the interaction occurring in the presence of the antibody is reduced by an amount that ranges from at least 30% to at least 99% relative to a control wherein the antibodies of this disclosure are absent.

In some embodiments, the antibodies of this disclosure inhibit C2 or C4-cleavage by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring C2 or C4-cleavage are well known in the art. The EC50 values for antibodies of this disclosure with respect C2 or C4-cleavage may be less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml. In some embodiments, the antibodies of this disclosure inhibit C2 or C4-cleavage at approximately equimolar concentrations of C1q and the respective anti-C1q antibody.

In some embodiments, the antibodies of this disclosure inhibit autoantibody-dependent and complement-dependent cytotoxicity (CDC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. The EC50 values for antibodies of this disclosure with respect to inhibition of autoantibody-dependent and complement-dependent cytotoxicity may be less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml.

In some embodiments, the antibodies of this disclosure inhibit complement-dependent cell-mediated cytotoxicity (CDCC) by at least 20%, at least 30%, at least 40%, at least 50%, at least 60%, at least 70%, at least 80%, at least 90%, at least 95%, or at least 99%, or by an amount that ranges from at least 30% to at least 99%, relative to a control wherein the antibodies of this disclosure are absent. Methods for measuring CDCC are well known in the art. The EC₅₀ values for antibodies of this disclosure with respect CDCC inhibition may be 1 less than 3 μg/ml; 2.5 μg/ml; 2.0 μg/ml; 1.5 μg/ml; 1.0 μg/ml; 0.5 μg/ml; 0.25 μg/ml; 0.1 μg/ml; 0.05 μg/ml. In some embodiments, the antibodies of this disclosure inhibit CDCC but not antibody-dependent cellular cytotoxicity (ADCC).

Humanized Anti-Complement C1q Antibodies (Incorporated by Reference from U.S. Pat. No. 10,316,081)

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat C1q.

In some embodiments, the human heavy chain constant region is a human IgG4 heavy chain constant region comprising the amino acid sequence of SEQ ID NO:47, or with at least 70%, at least about 75%, at least about 80%, at least about 85%, at least about 90% homology to SEQ ID NO: 47. The human IgG4 heavy chain constant region may comprise an Fc region with one or more modifications and/or amino acid substitutions according to Kabat numbering. In such cases, the Fc region comprises a leucine to glutamate amino acid substitution at position 248 (corresponding to L115E mutation in IgG4), wherein such a substitution inhibits the Fc region from interacting with an Fc receptor. In some embodiments, the Fc region comprises a serine to proline amino acid substitution at position 241 (corresponding to S108P in IgG4), wherein such a substitution prevents arm switching in the antibody.

The amino acid sequence of human IgG4 (S241P L248E; that is corresponding to S108P and L115E in SEQ ID NO: 47) heavy chain constant domain is:

(SEQ ID NO: 47)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38.

The amino acid sequence of heavy chain variable domain variant 1 (VH1) is:

(SEQ ID NO: 31)
QVQLVQSGAELKKPGASVKVSCKSSGYHFTSYWMHWVKQAPGQGLEWIG
VIHPNSGSINYNEKFESKATITVDKSTSTAYMQLSSLTSEDSAVYYCAG
ERDSTEVLPMDYWGQGTSVTVSS.

The hyper variable regions (HVRs) of VH1 are depicted in bolded and underlined text.

The amino acid sequence of heavy chain variable domain variant 2 (VH2) is:

(SEQ ID NO: 32)
QVQLVQSGAELKKPGASVKVSCKSSGYHFTSYWMHWVKQAPGQGLEWIG
VIHPNSGSINYNEKFESRATITVDKSTSTAYMELSSLRSEDTAVYYCAG
ERDSTEVLPMDYWGQGTTVTVSS.

The hyper variable regions (HVRs) of VH2 are depicted in bolded and underlined text.

The amino acid sequence of heavy chain variable domain variant 3 (VH3) is:

(SEQ ID NO: 33)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSS.

The hyper variable regions (HVRs) of VH3 are depicted in bolded and underlined text.

The amino acid sequence of heavy chain variable domain variant 4 (VH4) is:

(SEQ ID NO: 34)
QVQLVQSGAELKKPGASVKVSCKSSGYHFTSYWMHWVRQAPGQGLEWIG
VIHPNSGSINYNEKFESRVTITVDKSTSTAYMELSSLRSEDTAVYYCAG
ERDSTEVLPMDYWGQGTTVTVSS.

The hyper variable regions (HVRs) of VH4 are depicted in bolded and underlined text.

The amino acid sequence of kappa light chain variable domain variant 1 (Vκ1) is:

(SEQ ID NO: 35)
DVQITQSPSYLAASLGERATINCRASKSINKYLAWYQQKPGKTNKLLIY
SGSTLQSGIPARFSGSGSGTDFTLTISSLEPEDFAMYYCQQHNEYPLTF
GQGTKLEIK.

The hyper variable regions (HVRs) of Vκ1 are depicted in bolded and underlined text.

The amino acid sequence of kappa light chain variable domain variant 2 (Vκ2) is:

(SEQ ID NO: 36)
DVQITQSPSSLSASLGERATINCRASKSINKYLAWYQQKPGKANKLLIY
SGSTLQSGIPARFSGSGSGTDFTLTISSLEPEDFAMYYCQQHNEYPLTF
GQGTKLEIK.

The hyper variable regions (HVRs) of Vκ2 are depicted in bolded and underlined text.

The amino acid sequence of kappa light chain variable domain variant 3 (Vκ3) is:

(SEQ ID NO: 37)
DVQITQSPSSLSASLGERATINCRASKSINKYLAWYQQKPGK
APKLLIYSGSTLQSGIPARFSGSGSGTDFTLTISSLEPEDFA
MYYCQQHNEYPLTFGQGTKLEIK.

The hyper variable regions (HVRs) of Vκ3 are depicted in bolded and underlined text.

The amino acid sequence of kappa light chain variable domain variant 4 (Vκ4) is:

(SEQ ID NO: 38)
DIQLTQSPSSLSASLGERATINCRASKSINKYLAWYQQKPGK
APKLLIYSGSTLQSGIPARFSGSGSGTDFTLTISSLEPEDFA
MYYCQQHNEYPLTFGQGTKLEIK.

The hyper variable regions (HVRs) of Vκ4 are depicted in bolded and underlined text.

The antibody may comprise a light chain variable domain amino acid sequence that is at least 8500, 90%, or 9500 identical to SEQ ID NO:35-38 while retaining the HVR-L1 RASKSINTKYLA (SEQ ID NO:5), the HVR-L2 SGSTLQS (SEQ ID NO:6), and the HVR-L3 QQHINEYPLT (SEQ ID NO:7). The antibody may comprise a heavy chain variable domain amino acid sequence that is at least 85%, 90%, or 9500 identical to SEQ TD NO:31-34 while retaining the HVR-H1 GYHIFTSYWMH (SEQ ID NO:9), the HVR-H2 VIHIPNSGSINYNEKFES (SEQ ID NO:10), and the HVR-H3 ERDSTEVLPMDJY (SEQ TD NO:11).

The antibody may comprise a heavy chain and a light chain, wherein the heavy chain comprises the amino acid sequence of SEQ ID NO: 14; and the light chain comprises the amino acid sequence of SEQ TD NO: 40.

The amino acid sequence of the heavy chain is:

(SEQ ID NO: 14)
QVQLVQSGAELKKPGASVKVSCKSSGYHFTSYWMHWVKQAPGQ
GLEWIGVIHPNSGSINYNEKFESRVTITVDKSTSTAYMELSSL
RSEDTAVYYCAGERDSTEVLPMDYWGQGTTVTVSSASTKGPSV
FPLAPCSRSTSESTAALGCLVKDYFPEPVTVSWNSGALTSGVH
TFPAVLQSSGLYSLSSVVTVPSSSLGTKTYTCNVDHKPSNTKV
DKRVESKYGPPCPPCPAPEFEGGPSVFLFPPKPKDTLMISRTP
EVTCVVVDVSQEDPEVQFNWYVDGVEVHNAKTKPREEQFNSTY
RVVSVLTVLHQDWLNGKEYKCKVSNKGLPSSIEKTISKAKGQP
REPQVYTLPPSQEEMTKNQVSLTCLVKGFYPSDIAVEWESNGQ
PENNYKTTPPVLDSDGSFFLYSRLTVDKSRWQEGNVFSCSVMH
EALHNHYTQKSLSLSLGK.

The hyper variable regions (HVRs) of VH3 are depicted in bolded and underlined text.

The amino acid sequence of the light chain is:

(SEQ ID NO: 40)
DVQITQSPSSLSASLGERATINCRASKSINKYLAWYQQKPGKA
PKLLIYSGSTLQSGIPARFSGSGSGTDFTLTISSLEPEDFAMY
YCQQHNEYPLTFGQGTKLEIKRTVAAPSVFIFPPSDEQLKSGT
ASVVCLLNNFYPREAKVQWKVDNALQSGNSQESVTEQDSKDST
YSLSSTLTLSKADYEKHKVYACEVTHQGLSSPVTKSFNRGEC

The complementarity determining regions (CDRs) of SEQ ID NO:40 are depicted in bolded and underlined text.

In some embodiments, humanized anti-C1q antibodies of the present disclosure include a heavy chain variable region that contains a Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or IgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a positions corresponding to position 248 inhibits the Fc region from interacting with an Fc receptor. In some embodiments, the amino acid substitution at position 248 or a positions corresponding to position 248 is a leucine to glutamate amino acid substitution. In some embodiments, the amino acid substitution at position 241 or a positions corresponding to position 241 prevents arm switching in the antibody. In some embodiments, the amino acid substitution at position 241 or a positions corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

### Anti-C1q Fab Fragment

All anti-C1q antibody Fab fragment sequences are incorporated by reference from U.S. patent application Ser. No. 15/360,549, which is hereby incorporated by reference for the antibodies and related compositions that it discloses.

In certain embodiments, the present disclosure provides an anti-C1q antibody Fab fragment that binds to a C1q protein comprising a heavy (V_{H}/C_{H}1) and light chain (V_{L}/C_{L}), wherein the anti-C1q antibody Fab fragment has six complementarity determining regions (CDRs), three each from V_{L} and V_{H} (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence:

(SEQ ID NO: 39)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPS
SKSTSGGTAALGCLVKDYFPEPVTVSWNSGALTSGVHTFPAVLQS
SGLYSLSSVVTVPSSSLGTQTYICNVNHKPSNTKVDKKVEPKSCD
KTHT

The complementarity determining regions (CDRs) of SEQ ID NO:39 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKPGKAPK
LLIYSGSTLQSGIPARFSGSGSGTDFTLTISSLEPEDFAMYYCQQ
HNEYPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

The complementarity determining regions (CDRs) of SEQ ID NO:40 are depicted in bolded and underlined text.

### Anti-C1q Single-Arm Antibody

In certain aspects, the present disclosure provides an antibody that binds to a protein in the complement cascade, such as a C1q protein. The antibody that binds to C1q comprises a single C1q antigen-binding arm and an Fc region. The single C1q antigen-binding arm may comprise a light chain variable domain and a heavy chain variable domain. The Fc region may comprise a complex of a first and a second Fc polypeptide. The Fc region may comprise a Fc [symbol] receptor binding site mutation. The antibody may be of the IgG4 class. In some embodiments, one but not both of the Fc polypeptide is an N-terminally truncated heavy chain. In some embodiments, the Fc [symbol] receptor is Fc [symbol] RI, Fc [symbol] RII, or Fc [symbol] RIII, preferably Fc [symbol] RI. The Fc [symbol] receptor binding site mutation may comprise a IgG4 L115E mutation.

(SEQ ID NO: 40)
DVQITQSPSSLSASLGERATINCRASKSINKYLAWYQQKPGKAPK
LLIYSGSTLQSGIPARFSGSGSGTDFTLTISSLEPEDFAMYYCQQ
HNEYPLTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

The complementarity determining regions (CDRs) of SEQ ID NO: 40 are depicted in bolded and underlined text. In some embodiments, the HVR-L1 of the light chain variable domain has the sequence RASKSINKYLA (SEQ ID NO:5), the HVR-L2 of the light chain variable domain has the sequence SGSTLQS (SEQ ID NO:6), and the HVR-L3 of the light chain variable domain has the sequence QQHNEYPLT (SEQ ID NO:7).

One heavy chain of the single-arm antibody (the heavy chain 1 domain) of the single-arm antibody may comprise the following amino acid sequence (SEQ ID NO:2):

(SEQ ID NO: 2)
QVQLVQSGAELKKPGASVKVSCKSSGYHFTSYWMHWVKQAPGQGL
EWIGVIHPNSGSINYNEKFESRVTITVDKSTSTAYMELSSLRSED
TAVYYCAGERDSTEVLPMDYWGQGTTVTVSSASTKGPSVFPLAPC
SRSTSESTAALGCLVKDYFPEPVTVSWNSGALTSGVHTFPAVLQS
SGLYSLSSVVTVPSSSLGTKTYTCNVDHKPSNTKVDKRVESKYGP
PCPPCPAPEFEGGPSVFLFPPKPKDTLMISRTPEVTCVVVDVSQE
DPEVQFNWYVDGVEVHNAKTKPREEQFNSTYRVVSVLTVLHQDWL
NGKEYKCKVSNKGLPSSIEKTISKAKGQPREPQVYTLPPSQEEMT
KNQVSLWCLVKGFYPSDIAVEWESNGQPENNYKTTPPVLDSDGSF
FLYSRLTVDKSRWQEGNVFSCSVMHEALHNHYTQKSLSLSLGK

The complementarity determining regions (CDRs) of SEQ ID NO: 2 are depicted in bolded and underlined text. The knob in hole T366W mutation (corresponding to IgG4 T246W mutation) in SEQ ID NO: 2 is depicted in underlined text. The S241P (for IgG4 arm swapping, corresponding to S108P) and L248E (for FCR, corresponding to L115E mutation) mutations are depicted in bolded text. In some embodiments, the HVR-H1 of the heavy chain variable domain has the sequence GYHFTSYWMH (SEQ ID NO:9), the HVR-H2 of the heavy chain variable domain has the sequence VIHPNSGSINYNEKFES (SEQ ID NO:10), and the HVR-H3 of the heavy chain variable domain has the sequence ERDSTEVLPMDY (SEQ ID NO:11).

A second heavy chain of the single-arm antibody (the heavy chain 2 domain) of the single-arm antibody, which is N-terminally truncated, may comprise the following amino acid sequence (SEQ ID NO: 3):

(SEQ ID NO: 3)
ESKYGPPCPPCPAPEFEGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLSCAVKGFYPSDIAVEWESNGQPENNYKTTPPVL
DSDGSFFLVSRLTVDKSRWQEGNVFSCSVMHEALHNHYTQKSLSL
SLGK

There is no heavy chain variable domain and no CDRs in SEQ ID NO: 3. The knob in hole T366S/L368A/Y407V mutations in SEQ ID NO: 3 are depicted in underlined text. The S241P and L248E mutations are depicted in bolded text.

In some embodiments, the antibody that binds to C1q, comprising:
a light chain domain comprising the amino acid sequence of SEQ ID NO: 40;
a first heavy chain domain comprising the amino acid sequence of SEQ ID NO: 2; and
a second heavy chain domain comprising the amino acid sequence of SEQ ID NO: 3; the second heavy chain domain is an N-terminally truncated heavy chain.

### C1s Inhibitors

The inhibitor of the classical complement pathway may be a C1s inhibitor, such as a small molecule, an antibody, an aptamer, an antisense nucleic acid or a gene editing agent.

Exemplary C1s small molecule inhibitors are described in U.S. patent application Ser. No. 17/379,334, the contents of which are incorporated herein by reference.

### Anti-Complement C1s Antibodies

Suitable inhibitors include an antibody that binds complement C1s protein (i.e., an anti-complement C1s antibody, also referred to herein as an anti-C1s antibody and a C1s antibody) and a nucleic acid molecule that encodes such an antibody. Complement C1s is an attractive target as it is upstream in the complement cascade and has a narrow range of substrate specificity. Furthermore it is possible to obtain antibodies (for example, but not limited to, monoclonal antibodies) that specifically bind the activated form of C1s.

All sequences mentioned in the following two paragraphs are incorporated by reference from U.S. patent application Ser. No. 14/890,811, which is hereby incorporated by reference for the antibodies and related compositions that it discloses.

In certain aspects, disclosed herein are methods of administering an anti-C1s antibody. The antibody may be a murine, humanized, or chimeric antibody. In some embodiments, the light chain variable domain comprises HVR-L1, HVR-L2, and HVR-L3, and the heavy chain comprises HVR-H1, HVR-H2, and HVR-H3 of a murine anti-human C1s monoclonal antibody 5A1 produced by a hybridoma cell line deposited with ATCC on May 15, 2013 or progeny thereof (ATCC Accession No. PTA-120351). In other embodiments, the light chain variable domain comprises the HVR-L1, HVR-L2, and HVR-L3 and the heavy chain variable domain comprises the HVR-H1, HVR-H2, and HVR-H3 of a murine anti-human C1s monoclonal antibody 5C12 produced by a hybridoma cell line deposited with ATCC on May 15, 2013, or progeny thereof (ATCC Accession No. PTA-120352).

In some embodiments, antibodies specifically bind to and inhibit a biological activity of C1s or the C1s proenzyme, such as C1s binding to C1q, C1s binding to C1r, or C1s binding to C2 or C4. The biological activity may be a proteolytic enzyme activity of C1s, the conversion of the C1s proenzyme to an active protease, or proteolytic cleavage of C2 or C4. In certain embodiments, the biological activity is activation of the classical complement activation pathway, activation of antibody and complement dependent cytotoxicity, or CiF hemolysis.

All anti-C1s antibody sequences are incorporated by reference from U.S. Pat. No. 8,877,197, which is hereby incorporated by reference for the antibodies and related compositions that it discloses.

In some embodiments, an anti-C1s antibody of the present disclosure (e.g., a subject antibody that specifically binds an epitope in a complement C1s protein) comprises: a) a light chain region comprising CDRs selected from SEQ ID NO:15, SEQ ID NO:16, and SEQ ID NO:17; and b) a heavy chain region comprising CDRs selected from SEQ ID NO:18, SEQ ID NO:19, and SEQ ID NO:20. In some of these embodiments, the anti-C1s antibody includes a humanized VH and/or VL framework region.

SEQ ID NO: 15:
SSVSSSYLHWYQ;
SEQ ID NO: 16:
STSNLASGVP;
SEQ ID NO: 17:
HQYYRLPPIT;
SEQ ID NO: 18:
GFTFSNYAMSWV;
SEQ ID NO: 19:
ISSGGSHTYY;
SEQ ID NO: 20:
ARLFTGYAMDY.

In some embodiments, an anti-C1s antibody of the present disclosure comprises a light chain variable region comprising amino acid sequence SEQ ID NO:21.

In some embodiments, an anti-C1s antibody of the present disclosure comprises a heavy chain variable region comprising amino acid sequence SEQ ID NO:22.

(SEQ ID NO: 21)
DIVMTQTTAIMSASLGERVTMTCTASSSVSSSYLHWYQQKPGSS
PKLWIYSTSNLASGVPARFSGSGSGTFYSLTISSMEAEDDATYY
CHQYYRLPPITFGAGTKLELK
(SEQ ID NO: 22)
QVKLEESGGALVKPGGSLKLSCAASGFTFSNYAMSWVRQIPEKR
LEWVATISSGGSHTYYLDSVKGRFTISRDNARDTLYLQMSSLRS
EDTALYYCARLFTGYAMDYWGQGTSVT

In some embodiments, an anti-C1s antibody of the present disclosure comprises a light chain variable region comprising amino acid sequence SEQ ID NO:23.

In some embodiments, an anti-C1s antibody of the present disclosure comprises a heavy chain variable region comprising amino acid sequence SEQ ID NO:24.

(SEQ ID NO: 23)
QIVLTQSPATLSLSPGERATMSCTASSSVSSSYLHWYQQKPGKA
PKLWIYSTSNLASGVPSRFSGSGSGTDYTLTISSLQPEDFATYY
CHQYYRLPPITFGQGTKLEIK
(SEQ ID NO: 24)
EVQLVESGGGLVKPGGSLRLSCAASGFTFSNYAMSWVRQAPGKG
LEWVATISSGGSHTYYLDSVKGRFTISRDNSKNTLYLQMNSLRA
EDTALYYCARLFTGYAMDYWGQGTLVTVSS

In some embodiments, an anti-C1s antibody of the present disclosure comprises a light chain comprising amino acid sequence SEQ ID NO:25.

In some embodiments, an anti-C1s antibody of the present disclosure comprises a heavy chain comprising amino acid sequence SEQ ID NO:26.

Sutimlimab antibody comprises a light chain comprising amino acid sequence SEQ ID NO:25 and a heavy chain comprising amino acid sequence SEQ ID NO:26.

(SEQ ID NO: 25)
QIVLTQSPATLSLSPGERATMSCTASSSVSSSYLHWYQQKPGKAP
KLWIYSTSNLASGVPSRFSGSGSGTDYTLTISSLQPEDFATYYCH
QYYRLPPITFGQGTKLEIKRTVAAPSVFIFPPSDEQLKSGTASVV
CLLNNFYPREAKVQWKVDNALQSGNSQESVTEQDSKDSTYSLSST
LTLSKADYEKHKVYACEVTHQGLSSPVTKSFNRGEC
(SEQ ID NO: 26)
EVQLVESGGGLVKPGGSLRLSCAASGFTFSNYAMSWVRQAPGKGL
EWVATISSGGSHTYYLDSVKGRFTISRDNSKNTLYLQMNSLRAED
TALYYCARLFTGYAMDYWGQGTLVTVSSASTKGPSVFPLAPCSRS
TSESTAALGCLVKDYFPEPVTVSWNSGALTSGVHTFPAVLQSSGL
YSLSSVVTVPSSSLGTKTYTCNVDHKPSNTKVDKRVESKYGPPCP
PCPAPEFEGGPSVFLFPPKPKDTLMISRTPEVTCVVVDVSQEDPE
VQFNWYVDGVEVHNAKTKPREEQFNSTYRVVSVLTVLHQDWLNGK
EYKCKVSNKGLPSSIEKTISKAKGQPREPQVYTLPPSQEEMTKNQ
VSLTCLVKGFYPSDIAVEWESNGQPENNYKTTPPVLDSDGSFFLY
SRLTVDKSRWQEGNVFSCSVMHEALHNHYTQKSLSLSLGK

In some embodiments, an anti-C1s antibody of the present disclosure comprises: a) a light chain region comprising CDRs selected from SEQ ID NO:27, SEQ ID NO:28, and SEQ ID NO:17; and b) a heavy chain region comprising CDRs selected from SEQ ID NO:29, SEQ ID NO:30, and SEQ ID NO:31. In some of these embodiments, the anti-C1s antibody includes a humanized VH and/or VL framework region.

SEQ ID NO: 27:
TASSSVSSSYLH;
SEQ ID NO: 28:
STSNLAS;
SEQ ID NO: 17:
HQYYRLPPIT;
SEQ ID NO: 29:
NYAMS;
SEQ ID NO: 30:
TISSGGSHTYYLDSVKG;
SEQ ID NO: 43:
LFTGYAMDY.

In some embodiments, an anti-C1s antibody of the present disclosure comprises a light chain variable region comprising amino acid sequence SEQ ID NO:44.

In some embodiments, an anti-C1s antibody of the present disclosure comprises a heavy chain variable region comprising amino acid sequence SEQ ID NO:45.

(SEQ ID NO: 45)
QIVLTQSPAIMSASLGERVTMTCTASSSVSSSYLHWYQQKPGS
SPKLWIYSTSNLASGVPARFSGSGSGTFYSLTISSMEAEDDAT
YYCHQYYRLPPITFGAGTKLELK
(SEQ ID NO: 44)
EVMLVESGGALVKPGGSLKLSCAASGFTFSNYAMSWVRQIPEK
RLEWVATISSGGSHTYYLDSVKGRFTISRDNARDTLYLQMSSL
RSEDTALYYCARLFTGYAMDYWGQGTSVTVSS

The anti-C1s antibody may be selected from an antigen binding fragment, Ig monomer, a Fab fragment, a F(ab′)₂ fragment, a Fd fragment, a scFv, a scAb, a dAb, a Fv, a single domain heavy chain antibody, a single domain light chain antibody, a mono-specific antibody, a bi-specific antibody, or a multi-specific antibody.

Disclosed herein are methods of administering an antibody that competes for binding the epitope bound by antibody IPN003 (also referred to herein as “IPN-M34” or “M34” or “TNT003”), e.g., an antibody comprising a variable domain of antibody IPN003, such as antibody IPN003.

### C1r Inhibitors

The inhibitor of the classical complement pathway may be a C1r inhibitor, such as a small molecule, an antibody, an aptamer, an antisense nucleic acid or a gene editing agent.

The anti-C1r antibodies disclosed herein are potent inhibitors of C1r.

The anti-C1r antibodies disclosed herein inhibit the interaction between C1r and C1q or between C1r and C1s, or wherein the anti-C1r antibody inhibits the catalytic activity of C1r or inhibits the processing of pro-C1r to an active protease.

### C1 Complex Inhibitors

The inhibitor of the classical complement pathway may be a C1 complex inhibitor, such as a small molecule, an antibody, an aptamer, an antisense nucleic acid or a gene editing agent.

The anti-C1 complex antibodies disclosed herein are potent inhibitors of C1 complex.

The anti-C1 complex antibodies disclosed herein inhibit C1r or C1s activation or blocks their ability to act on C2 or C4. The anti-C1 complex antibodies disclosed herein bind to a combinatorial epitope within the C1 complex, wherein said combinatorial epitope comprises amino acids of both C1q and C1s; both C1q and C1r; both C1r and C1s; or each of C1q, C1r, and C1s.

### C2 Inhibitors

The inhibitor of the classical complement pathway may be a C2 inhibitor, such as a small molecule, an antibody, an aptamer, an antisense nucleic acid or a gene editing agent.

The anti-C2 inhibitors disclosed herein are potent inhibitors of C2.

### C3 Inhibitors

The inhibitor of the classical complement pathway may be a C3 inhibitor, such as a small molecule, an antibody, an aptamer, an antisense nucleic acid or a gene editing agent.

The anti-C3 inhibitors disclosed herein are potent inhibitors of C3. In some embodiments, the C3 inhibitor is APL-9 (Apellis) and/or AMY-101 (Amyndas) and/or IVT CB 2782-PEG (Catalyst Biosciences and Biogen).

### C4 Inhibitors

The inhibitor of the classical complement pathway may be a C4 inhibitor, such as a small molecule, an antibody, an aptamer, an antisense nucleic acid or a gene editing agent.

The anti-C4 inhibitors disclosed herein are potent inhibitors of C4.

### Inhibition of Complement

A number of molecules are known that inhibit the activity of complement. In addition to known compounds, suitable inhibitors can be screened by methods described herein. As described above, normal cells can produce proteins that block complement activity, e.g., CD59, C1 inhibitor, etc. In some embodiments of the disclosure, complement is inhibited by upregulating expression of genes encoding such polypeptides.

Modifications of molecules that block complement activation are also known in the art. For example, such molecules include, without limitation, modified complement receptors, such as soluble CR1. The mature protein of the most common allotype of CR1 contains 1998 amino acid residues: an extracellular domain of 1930 residues, a transmembrane region of 25 residues, and a cytoplasmic domain of 43 residues. The entire extracellular domain is composed of 30 repeating units referred to as short consensus repeats (SCRs) or complement control protein repeats (CCPRs), each consisting of 60 to 70 amino acid residues. Recent data indicate that C1q binds specifically to human CR1. Thus, CR1 recognizes all three complement opsonins, namely C3b, C4b, and C1q. A soluble version of recombinant human CR1 (sCR1) lacking the transmembrane and cytoplasmic domains has been produced and shown to retain all the known functions of the native CR1. The cardioprotective role of sCR1 in animal models of ischemia/reperfusion injury has been confirmed. Several types of human C1q receptors (C1qR) have been described. These include the ubiquitously distributed 60- to 67-kDa receptor, referred to as cC1qR because it binds the collagen-like domain of C1q. This C1qR variant was shown to be calreticulin; a 126-kDa receptor that modulates monocyte phagocytosis. gC1qR is not a membrane-bound molecule, but rather a secreted soluble protein with affinity for the globular regions of C1q, and may act as a fluid-phase regulator of complement activation.

Decay accelerating factor (DAF) (CD55) is composed of four SCRs plus a serine/threonine-enriched domain that is capable of extensive O-linked glycosylation. DAF is attached to cell membranes by a glycosyl phosphatidyl inositol (GPI) anchor and, through its ability to bind C4b and C3b, it acts by dissociating the C3 and C5 convertases. Soluble versions of DAF (sDAF) have been shown to inhibit complement activation.

C1 inhibitor, a member of the “serpin” family of serine protease inhibitors, is a heavily glycosylated plasma protein that prevents fluid-phase C1 activation. C1 inhibitor regulates the classical pathway of complement activation by blocking the active site of C1r and C1s and dissociating them from C1q.

Peptide inhibitors of complement activation include C5a and other inhibitory molecules include Fucan.

### Methods of Treatment

The present disclosure presents a model evaluating complement activation, alone or in combination with NfL and age, that more accurately predicts HD stage than NfL alone or in combination with age. Data from HD clinical trials demonstrate that patients identified via the use of a model evaluating high C4a levels and/or high C4a/C4 ratios exhibit clinical improvement with anti-C1q antibody treatment. The clinical trial data also demonstrates treatment with full-length anti-C1q antibody leads to clinical benefits in HD patients identified by the use of a model evaluating complement activation alone or in combination with NfL and age. The clinical trial data also demonstrate that accounting for complement activation, NfL, and age, and the use of a full-length anti-C1q antibody reliably leads to clinical benefits in HD patients. Clinical benefits were measured by composite Unified Huntington's Disease Rating Scale (cUHDRS), or any of the following domains: cognitive (SDMT, SWR), total functional capacity (TFC), and total motor skills (TMS).

The present disclosure is generally directed to methods of treating Huntington's disease in a subject in need thereof. The method comprises determining that the subject has an elevated level of C4a or an elevated C4a/C4 ratio; and administering to the subject an inhibitor of the classical complement pathway, e.g., if the subject has an elevated level of C4a or an elevated C4a/C4 ratio. A therapeutically effective amount of the inhibitor may be administered.

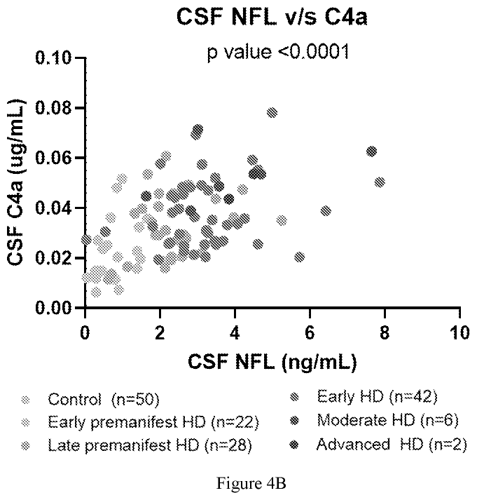

The elevated level of C4a may be greater than a C4a level in normal or healthy subjects, such as subjects of a similar age. Patients treated with inhibitor of the classical complement pathway (e.g., full-length C1q antibody) showed improvement from baseline in cUHDRS and/or components of cUHDRS over 6 months of treatment. This improvement was enriched in patients with excess complement activity at baseline, whereby 75% of patients with elevated CSF C4a levels at baseline demonstrated improvement in cUHDRS over 6 months of treatment, suggesting rapid response to anti-C1q therapy potentially via enhanced synapse function. C4a is an objective measurement that can indicate excess classical complement activity in CSF that correlates with disease stage and multiple clinical end-points in HD. These findings support the potential for enrichment strategies for patients with elevated C4a and excess complement activity who may benefit from anti-C1q therapy. C4a can inform disease stage beyond predicted by age (FIG. 5). Data below the diagonal line in FIG. 5 indicates that LM2 (age+C4a) has lower error, hence higher accuracy, than LM1 (age alone).

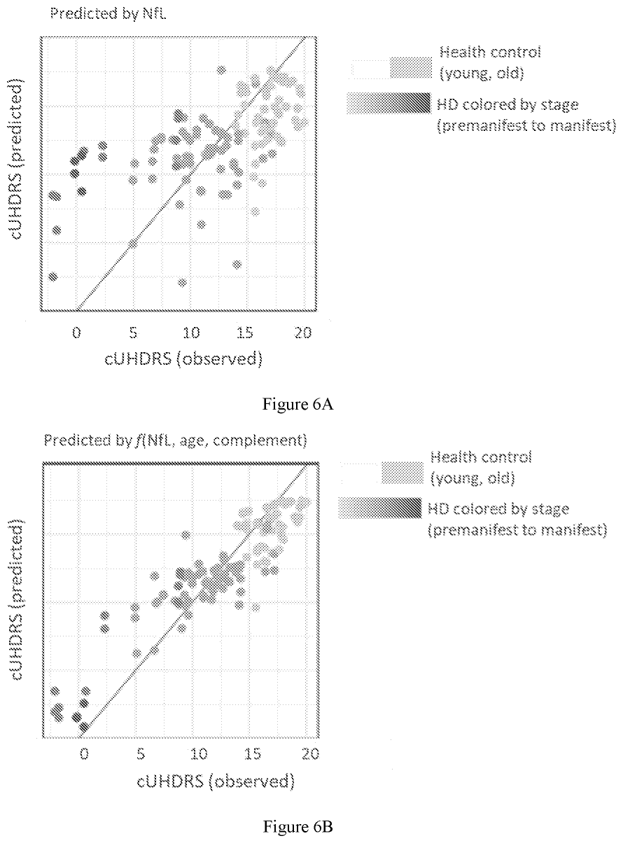

The elevated level of C4a may be greater than a reference C4a level. For example, the reference C4a level is a value that is equal to or greater than the median of C4a levels in samples derived from Huntington's disease subjects, such as subjects of a similar age. In other embodiments, the reference C4a level is a value that is equal to or greater than the 75th percentile of C4a levels in samples derived from normal or healthy subjects (subjects that do not have Huntington's disease.), such as subjects of a similar age. Approximately 50% of HD patients have elevated CSF levels of complement activation product C4a in natural history cohorts (FIGS. 7A-7B). The median of the C4a levels in samples derived from Huntington's disease subjects is similar to the 75th percentile of C4a levels in samples derived from normal or healthy subjects (FIG. 7A).

In some embodiments, the elevated level of C4a is greater than the reference C4a level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%. In some embodiments, the elevated level of C4a is greater than the reference C4a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

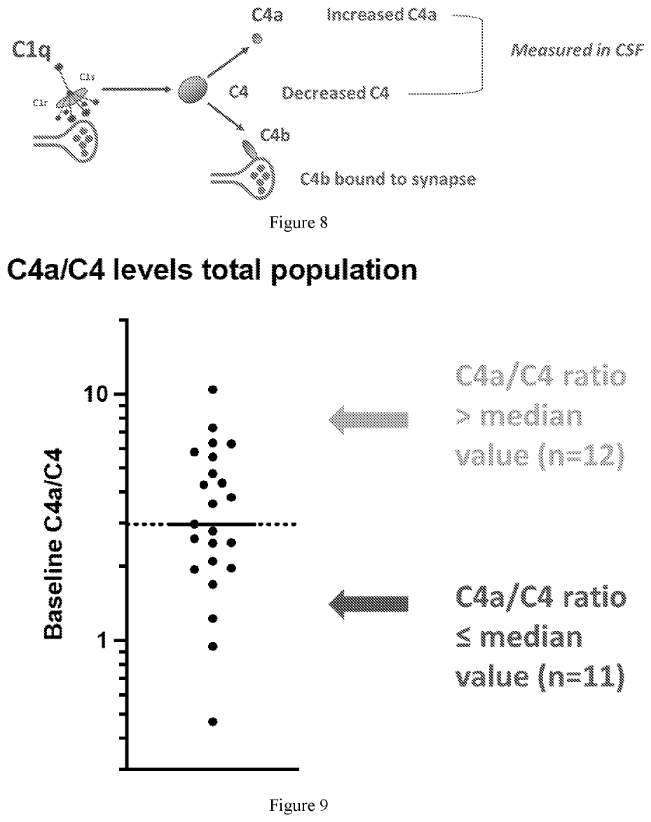

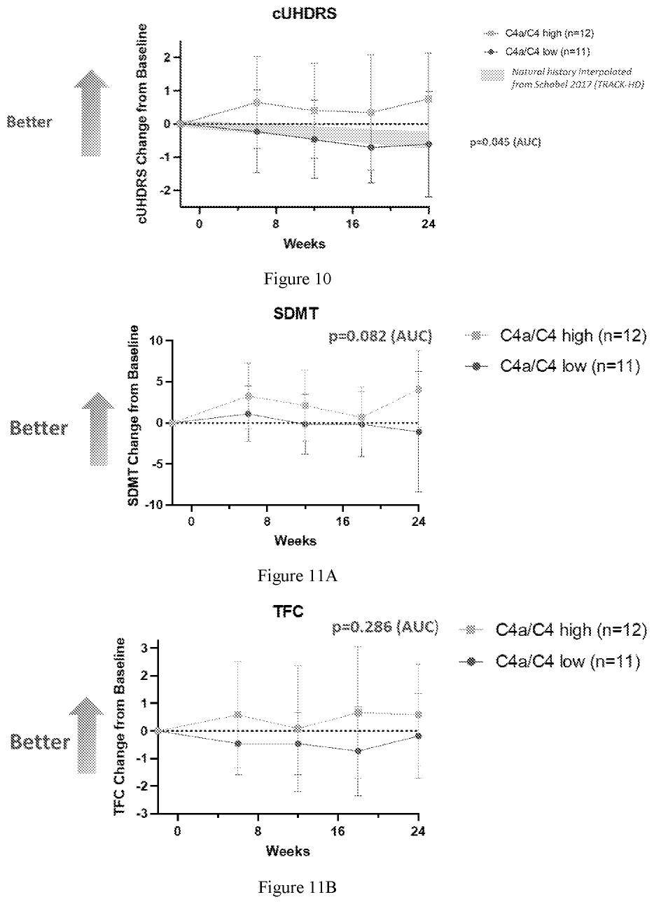

Patients were divided into two groups based on C4a/C4 ratio at baseline (FIG. 9). The baseline was at the median of the C4a/C4 ratio in samples derived from Huntington's disease subjects, which is similar to the 75th percentile of C4a/C4 ratio in samples derived from normal or healthy subjects. High baseline complement patients showed consistent and significant improvement on cUHDRS over 24-week treatment period (FIG. 10).

Accordingly, the elevated C4a/C4 ratio may be greater than a C4a/C4 ratio in normal or healthy subjects, such as subjects of a similar age. In some embodiments, the elevated C4a/C4 ratio is greater than a reference C4a/C4 ratio. The reference C4a/C4 ratio may be a value that is equal to or greater than the median of C4a/C4 ratio in samples derived from Huntington's disease subjects, such as subjects of a similar age. In other embodiments, the reference C4a/C4 ratio is a value that is equal to or greater than the 75th percentile of C4a/C4 ratios in samples derived from normal or healthy subjects (subjects that do not have Huntington's disease), such as subjects of a similar age.

In some embodiments, the elevated C4a/C4 ratio is greater than the reference C4a/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, or 500%. In some embodiments, the elevated C4a/C4 ratio is greater than the reference C4a/C4 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

In some embodiments, the level of C4a or the C4a/C4 ratio is measured in cerebrospinal fluid (CSF) or plasma. Any methods of measuring complement C4a and C4 levels can be used, and are well known to those of ordinary skill in the art, including but not limited to commercially available ELISA kits such as those from Quidel and SVAR.

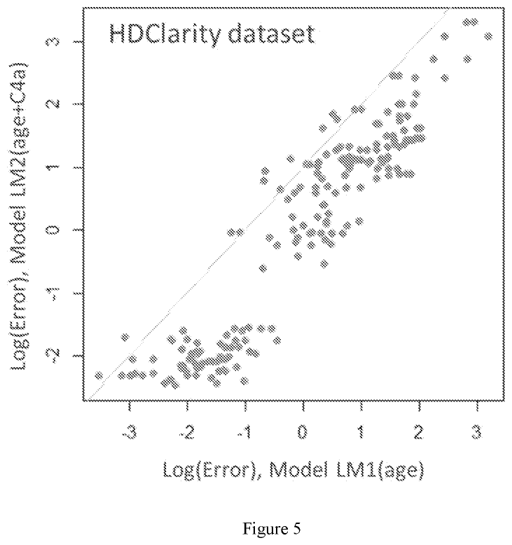

Neurofilament light (NfL) is a strong monitoring and prognostic biomarker for HD. However, a model combining complement activation, NfL and age more accurately predicts HD stage than NfL alone (FIGS. 6A-6B).

In some embodiments, the subject has an elevated level of Neurofilament light chain (NfL). The elevated level of NfL may be greater than a NfL level in normal or healthy subjects, such as subjects of a similar age. In some embodiments, the elevated level of NfL is greater than a reference NfL level. The reference NfL level may be about 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1100 pg/ml, 1200 pg/ml, 1300 pg/ml, 1400 pg/ml, 1500 pg/ml, 1600 pg/ml, 1700 pg/ml, 1800 pg/ml, 1900 pg/ml, or 2000 pg/ml. In some embodiments, the elevated level of NfL is greater than the NfL level in normal or healthy subjects or the reference NfL level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, or 5000%. In some embodiments, the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%. In some embodiments, the level of NfL is measured in cerebrospinal fluid (CSF).

In other embodiments, the level of NfL is measured in plasma. The reference NfL level may be about 1 pg/ml, 2 pg/ml, 3 pg/ml, 4 pg/ml, 5 pg/ml, 6 pg/ml, 7 pg/ml, 8 pg/ml, 9 pg/ml, 10 pg/ml, 11 pg/ml, 12 pg/ml, 13 pg/ml, 14 pg/ml, 15 pg/ml, 16 pg/ml, 17 pg/ml, 18 pg/ml, 19 pg/ml, or 20 pg/ml. In some embodiments, the elevated level of NfL is greater than the NfL level in normal or healthy subjects or the reference NfL level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 1500%, 2000%, 2500%, 3000%, 3500%, 4000%, 4500%, or 5000%. In some embodiments, the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%.

In some embodiments, the inhibitor of the classical complement pathway is a C1q inhibitor, such as a small molecule, an antibody, an aptamer, an antisense nucleic acid or a gene-editing agent. The antibody may be a C1q antibody described herein. For example, the antibody may comprise a heavy chain and a light chain, wherein the heavy chain comprises the amino acid sequence of SEQ ID NO: 14; and the light chain comprises the amino acid sequence of SEQ ID NO: 40. Any suitable dosing may be used in the methods described herein. For example, the antibody may be administered at a dose of at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg. In some embodiments, the antibody is administered at a dose of 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 105 mg/kg, 110 mg/kg, 120 mg/kg, 130 mg/kg, 140 mg/kg, or 150 mg/kg. In some embodiments, the antibody is administered at a dose of 75 mg/kg on day 1 and on day 5 or day 6. In some embodiments, the antibody is further administered at a dose of 100 mg/kg every two weeks.

In some embodiments, the antibody is administered intravenously. In some embodiments, the antibody is administered once a week, once every other week, once a month, once every six weeks, or once every other month. In some embodiments, the antibody is administered for at least 3 months, at least 6 months, at least 7 months, at least 8 months, at least 9 months, at least 10 months, at least 11 months, or at least 12 months. In some embodiments, the antibody is administered for 3 months, 6 months, 7 months, 8 months, 9 months, 10 months, 11 months, or 12 months.

In some embodiments, the antibody comprises a heavy chain and a light chain, wherein the heavy chain comprises the amino acid sequence of SEQ ID NO: 14; and the light chain comprises the amino acid sequence of SEQ ID NO: 40. The antibody may be administered at a dose of 75 mg/kg on day 1 and on day 5 or day 6. In some embodiments, the antibody is further administered at a dose of 100 mg/kg every two weeks.

### EXAMPLES

### Example 1: Improved Model of Predicting HD Stage

Discovery and tested cohorts are patients at University College London (UCL) (n=60) and HDClarity (n=100), respectively. Complement proteins and NfL were measured using in-house ELISA and Uman kit, respectively. A machine learning model was developed to assess contribution of complement activation towards cUHDRS. Accuracy and improvement of this model over linear models of NfL and age were determined by root mean square error.

C4 was measured by sandwich ELISA. Plates were coated with 3 ug/ml polyclonal raised to C4 (Goat anti-Human C4, Complement Tech, A205). CSF was diluted 1:5000 in dPBS (Delbecco's PBS) buffer containing 10 mM EDTA and incubated overnight at 4-degree C. Plates were washed and incubated with antibody anti-C4 antibody (Abcam ab47788) conjugated to Alkaline phosphatase (1:2000 dilution). Plates were washed thrice with wash buffer and developed using 75 uL of alkaline phosphatase substrate (Life Technologies, T2214). After 20 minutes at room temperature, plates were read using a luminometer. Standards were fit using a 4PL logistic fit and concentration of unknowns determined. Analyte levels were corrected for dilution and then plotted using GraphPad Prism.

C4a was measured in a competition-based ELISA. Plates were coated with 10 ug/ml CT-C4a (Rabbit anti human C4a, Complement Tech A206), a polyclonal antisera raised to C4a. CSF was diluted 1:10 in buffer dPBS containing 10 mM EDTA and 10 ng/ml biotinylated-C4a and incubated overnight at 4 degree C. Plates were washed 3 times and incubated with Avidin-AP (1:1000 dilution). Alkaline phosphates activity was detected as above.

Patients experienced improvements in clinical outcomes, as measured by Composite UHDRS (cUHDRS), a clinical rating scale used to assess four domains of clinical performance and capacity in HD including motor function, cognitive function, behavioral abnormalities and functional capacity. cUHDRS is a primary endpoint in HD consisting of 4 sub-domains assessing HD disease progression (cognition, motor, and function).

Overall, patients maintained clinical function with no decline from baseline in cUHDRS over 6 months of treatment (n=23), which compares to natural history data showing that HD patients experience a decline of approximately 1.2 points over one year or 0.6 points over six months. Additionally, 56% of patients showed improvement from baseline in cUHDRS and all subdomains of cUHDRS over 6 months of treatment. This improvement was enriched in patients with excess complement activity at baseline, whereby 75% of patients with elevated CSF C4a levels at baseline demonstrated improvement in cUHDRS over 6 months of treatment, suggesting rapid response to anti-C1q therapy potentially via enhanced synapse function. C4a is an objective measurement of excess classical complement activity in CSF that correlates with disease stage and multiple clinical endpoints in HD. These findings support the potential for enrichment strategies for patients with elevated C4a and excess complement activity who may benefit from anti-C1q therapy.

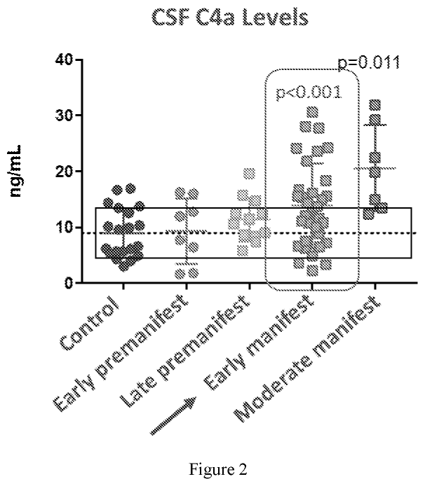

Excess classical complement activity in CSF (C4a) of HD patients is associated with functional decline and disease severity (FIG. 2). Further, CSF C4a is correlated with individual domains of the cUHDRS (Table 1).

| Clinical endpoints | p-value | function |
|---|---|---|
| Total functional score (TFC) | 0.0333 | Motor & Function |
| Total motor score (TMS) | 0.0181 | Motor & Function |
| Disease burden score (DBS) | 0.1310 | Motor & Function |
| Symbol digit mod. Test | 0.0324 | Cognitive Scales |
| (SDMT) | | |
| Verbal fluency | 0.0255 | Cognitive Scales |
| Stroop color naming (SCN) | 0.0454 | Cognitive Scales |
| Stroop word recall (SWR) | 0.0710 | Cognitive Scales |

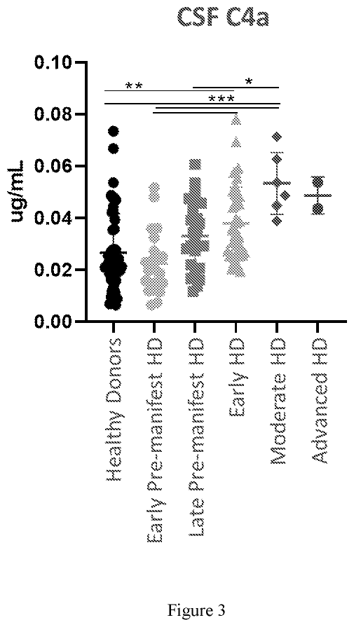

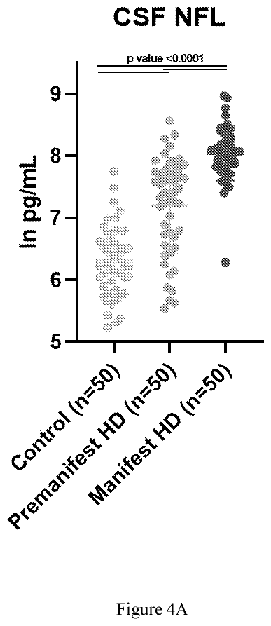

C4a increases in the CSF of manifest HD patients (FIG. 3). NFL is significantly elevated in CSF of manifest HD patients and CSF NFL correlates with CSF C4a (FIGS. 4A-4B).

Using the HDClarity dataset, several machine learning models were built to determine if complement factors would aid understanding of patient's clinical assessment in manifested HD when increase in NfL plateaus. Consistent with earlier reports, NfL and age were found to be informative of predicting patients' clinical severity scores. In addition, the models suggested that proteins involved in the activation of the classical complement pathway, C4a and C4, were the next important biomarkers towards predicting both cUHDRS as well as the individual clinical tests. These results affirm a likely importance for the complement pathway in HD progression and suggest these proteins could have value in modelling and predicting disease stage and therapeutic effects. Furthermore, these models could serve as quantitative and sensitive tools to evaluate therapeutic effects in short studies when changes in conventional functional assessments are difficult to accurately determine.

C4a can inform disease stage beyond predicted by age (FIG. 5). To determine if C4a offers additional information about disease stage beyond age alone, we assessed the statistical significance of C4a coefficient towards predicting HD disease stage. Two linear regression models were used to compare correlation between age and age+C4a with HD category. LM2, which includes C4a in addition to age, predicts HD disease stage more accurately compared to LM1. The diagonal line represents equal accuracy between two models. Data below the diagonal line indicates that LM2 has lower error, hence higher accuracy, than LM1 (FIG. 5). The contribution of C4a is statistically significant (p=0.007), indicating that C4a levels in HD patients is informative of their stage beyond what is expected as an aging effect.

A model combining complement activation, NfL and Age more accurately predicts HD stage than NfL alone (FIGS. 6A-6B).

Approximately 50% of HD patients have elevated CSF levels of complement activation product C4a in natural history cohorts. Similar range in current study—these patient may respond better to anti-C1q therapy (FIGS. 7A-7B).

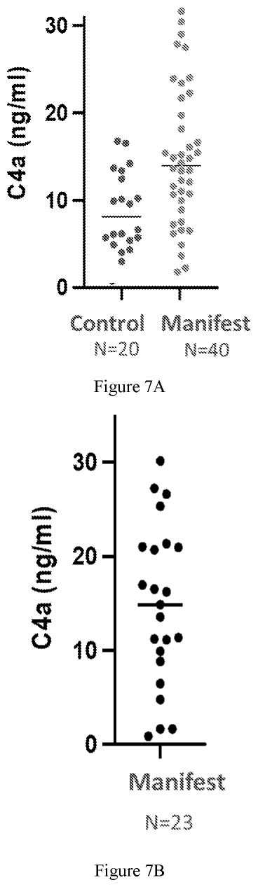

CSF C4a/C4 ratio: a sensitive measure of ongoing complement activation. C4 decreased (consumed) with C1q activation activity. C4a increased (produced) with C1q activation. C4a/C4 ratio corrects for genetic variability among subjects FIG. 8).

### Example 2: A Phase 2a Open Label Study to Assess the Safety, Tolerability, Pharmacokinetics and Pharmacodynamics of Intravenous Full-Length C1q Antibody in Subjects with, or at Risk for, Manifest Huntington's Disease

### Overview

Subjects received induction dosing of full-length C1q antibody (the antibody comprises the heavy chain comprising the amino acid sequence of SEQ ID NO: 14; and the light chain comprising the amino acid sequence of SEQ ID NO: 40) at 75 mg/kg administered by intravenous (IV) infusion on Days 1 and 5 or 6, followed by maintenance dosing at 100 mg/kg every 2 weeks (Weeks 2, 4, 6, 8, 10, 12, 14, 16, 18, 20, and 22) with follow up visits on Weeks 24, 28, and 36. All full-length C1q antibody infusions were administered at in clinic visits.

All subjects were contacted 6 months after study completion to gather information focused towards clinical signs and symptoms suggestive of autoimmunity, e.g., fatigue, muscle aches, swelling, redness, low-grade fever, trouble concentrating, numbness and tingling, hair loss, or skin rashes.

Blood samples for PK/PD assessments were collected for serial sampling following the first and last dose. Pre-dose samples were collected at all dosing visits (Days 1 and 5/6 and Weeks 2 through 22) prior to beginning the full-length C1q antibody infusion. Blood samples were collected after completing the infusion on Day 5/6, and Weeks 2, 6, 10, 14, and 18. PK/PD samples were also collected at the Week 24, 28, and 36 visits.

Cerebrospinal fluid (CSF) sampling for PK and PD assessments were conducted at Screening, pre-dose at Week 6 and Week 12, and at Weeks 24 and 36.

EEGs were conducted during Screening and pre-dose on Day 1, Week 6, Week 12, and Week 18, as well as at Weeks 24 and 36.

In the Phase 2 trial, safety was assessed in all 28 patients enrolled. Initial target engagement data included pharmacokinetics (PK) and pharmacodynamics (PD) in 17 patients who completed the 24-week treatment period. Initial efficacy and biomarker data included clinical outcomes, as measured by the Unified Huntington's Disease Rating Scale (UHDRS) in all 23 patients who completed the 24-week treatment period, as well as neurofilament light chain (NfL) levels in 16 patients who completed the 24-week treatment period. C4a, C4, and Nfl levels were measured as described in Example 1. Initial findings show that treatment with full-length C1q antibody was generally well-tolerated, with robust target engagement of C1q in both serum and cerebrospinal fluid (CSF) through the dosing period. Notably, meaningful improvements in UHDRS were observed, while NfL levels in both plasma and CSF remained generally unchanged and consistent with HD natural history.

The initial data on full-length C1q antibody suggest that protection of functioning synapses via C1q inhibition can lead to rapid functional improvement in HD, and potentially other neurodegenerative diseases. The signals of clinical response observed, particularly in patients with elevated C4a, also suggest that patients with excess complement activity may respond better and more quickly to anti-C1q therapy.

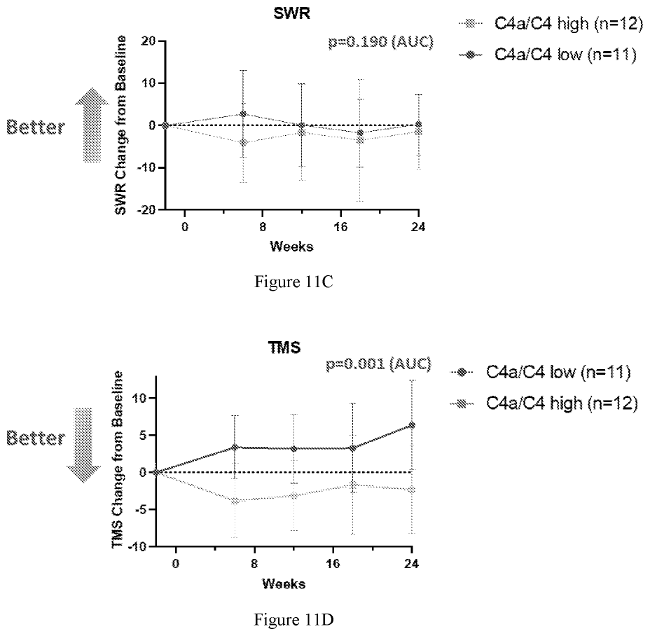

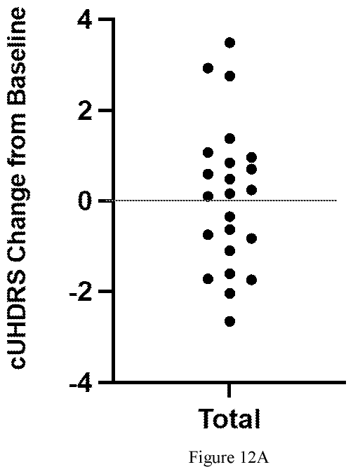

The phase 2a data was analyzed using CSF C4a/C4 ratio as measure of ongoing complement activity (FIG. 9). Patients were divided into two groups based on C4a/C4 levels at baseline. High baseline complement patients showed consistent and significant improvement on cUHDRS over 24-week treatment period (FIG. 10). Biomarker-differentiated responses are unlikely to be placebo driven. The high baseline C4a/C4 ratio patients also improved from baseline on cUHDRS over 24-week treatment period vs. natural history. The decline of low complement activity cohort is consistent with natural history. Further, high baseline C4a/C4 ratio patients consistently improved on cUHDRS sub-domains over 24-week treatment period (FIGS. 11A-11D). 75% of patients with high baseline complement activity (C4a/C4) improved at week 24 in cUHDRS vs. 36% with low activation (FIGS. 12A-12B).

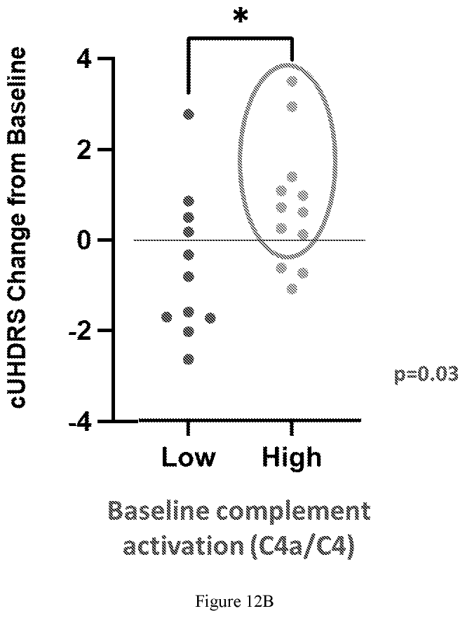

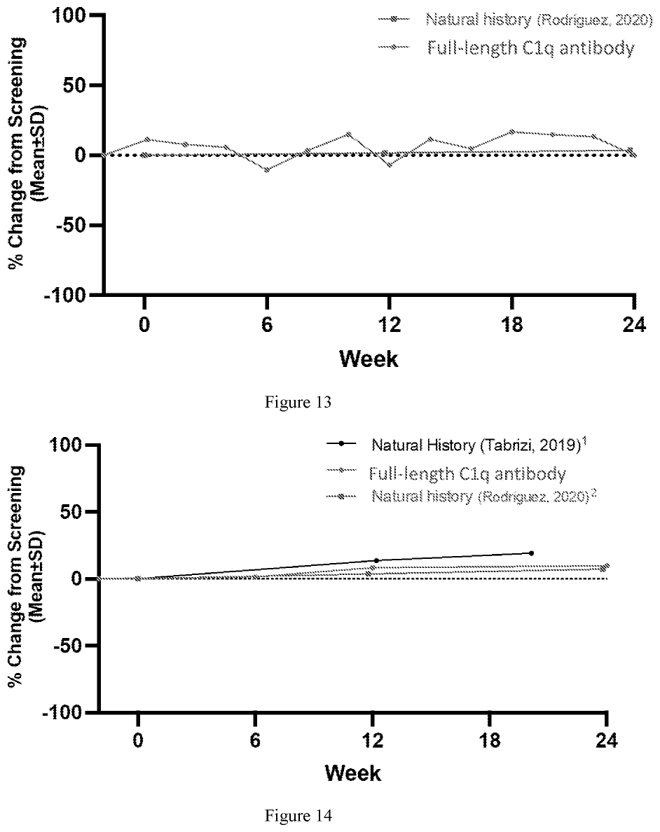

Plasma NfL levels are consistent with HD natural history at week 24 (FIG. 13). CSF NfL levels are also consistent with HD natural history at week 24 (FIG. 14). The HD natural history data are for manifest cohort (Rodriguez, et al., Sci Transl Med. 2020 12 16; 12(574)) and (Tabrizi, NEJM 2019, 38:2307). Synapse loss impacts neuronal function and precedes neuronal loss and NfL release (Ravalia, 2021; Milnerwood and Raymond, 2010; Milnerwood et al., 2010; Parsons and Raymond, 2014; Ravalia et al., 2021; Alirezaeil et al., 2020 Mol Neurobiol. 57: 469-491).

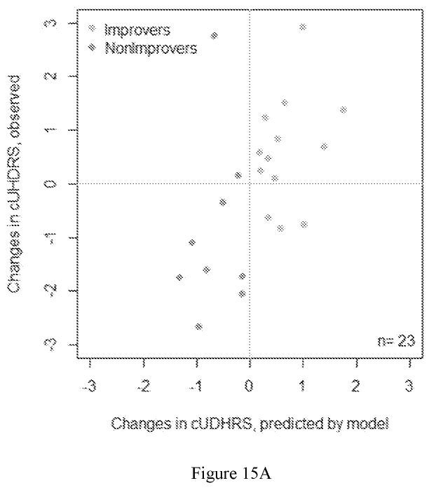

Combined baseline C4a ratio, NfL and age identifies separate groups of differential cUHDRS at week 24 (FIGS. 15A-15B). Improvers identified by model tend to have higher baseline C4a/C4, lower baseline NfL and slightly younger.

### Example 3: A Phase 2a Open Label Study to Assess the Effect of Intravenous Full-Length C1q Antibody in Subjects with, or at Risk for, Manifest Huntington's Disease

### Overview

Subjects received induction dosing of full-length C1q antibody (the antibody comprises the heavy chain comprising the amino acid sequence of SEQ ID NO: 14; and the light chain comprising the amino acid sequence of SEQ ID NO: 40) at 75 mg/kg administered by intravenous (IV) infusion on Days 1 and 5 or 6, followed by maintenance dosing at 100 mg/kg every 2 weeks (Weeks 2, 4, 6, 8, 10, 12, 14, 16, 18, 20, and 22) with follow up visits on Weeks 24, 28, and 36. All full-length C1q antibody infusions were administered at in clinic visits.

Blood samples for PK/PD assessments were collected for serial sampling following the first and last dose. Pre-dose samples were collected at all dosing visits (Days 1 and 5/6 and Weeks 2 through 22) prior to beginning the full-length C1q antibody infusion. Blood samples were collected after completing the infusion on Day 5/6, and Weeks 2, 6, 10, 14, and 18. PK/PD samples were also collected at the Week 24, 28, and 36 visits.

Cerebrospinal fluid (CSF) sampling for PK and PD assessments were conducted at Screening, pre-dose at Week 6 and Week 12, and at Weeks 24 and 36.

EEGs were conducted during Screening and pre-dose on Day 1, Week 6, Week 12, and Week 18, as well as at Weeks 24 and 36.

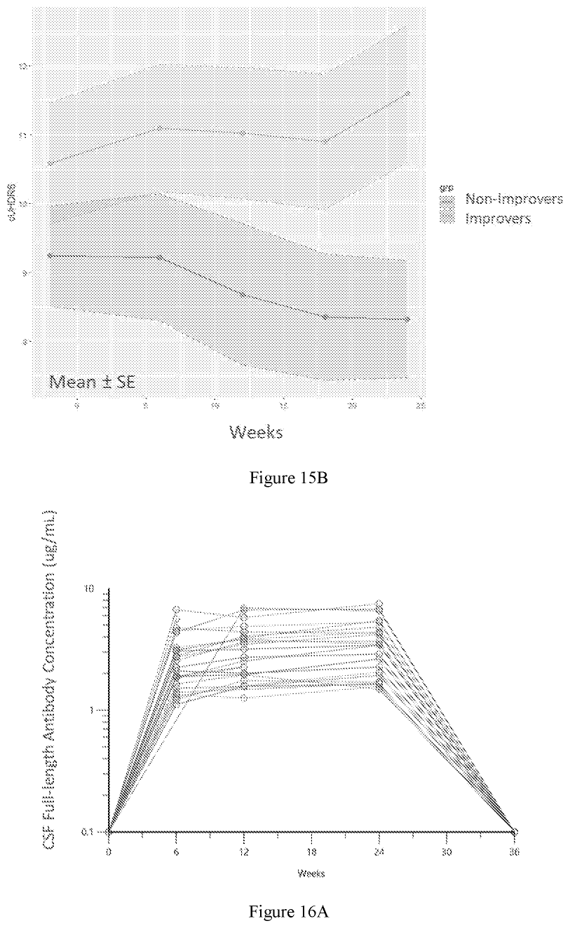

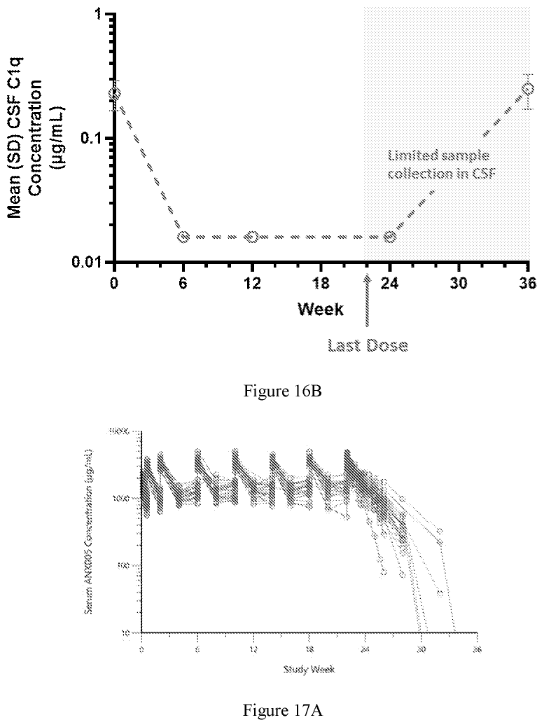

The initial data suggest that full-length C1q antibody demonstrated rapid, robust and long-lasting complement engagement. Full C1q engagement in CSF at first timepoint was assessed. FIG. 16A shows full-length C1q antibody levels in CSF while FIG. 16B shows full engagement of C1q in CSF. Full C1q engagement in serum was assessed upon first dose, and 6 to 10 weeks after full-length C1q antibody treatment was stopped. FIG. 17A shows full-length C1q antibody levels in serum while FIG. 17B shows full engagement of C1q in serum.

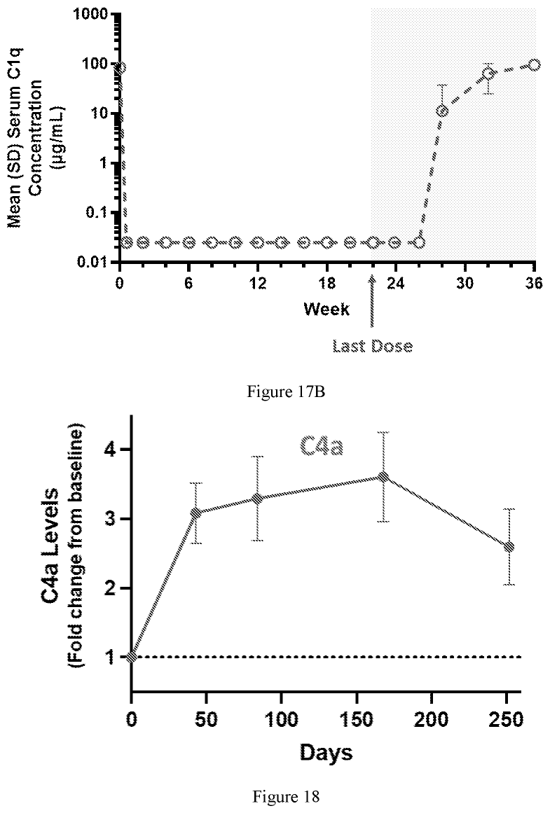

The data also shows extended target engagement and downstream complement inhibition within the CNS 14 weeks post last dose. FIG. 18 shows prolonged full-length C1q antibody effect throughout 3-month off-treatment period. C4a release is a pharmacodynamic marker of C1q target engagement. All patients have evidence of continued target engagement 3 months post last dose. This is consistent with preclinical data showing full-length C1q antibody accumulation at sites of high C1q expression in CNS.

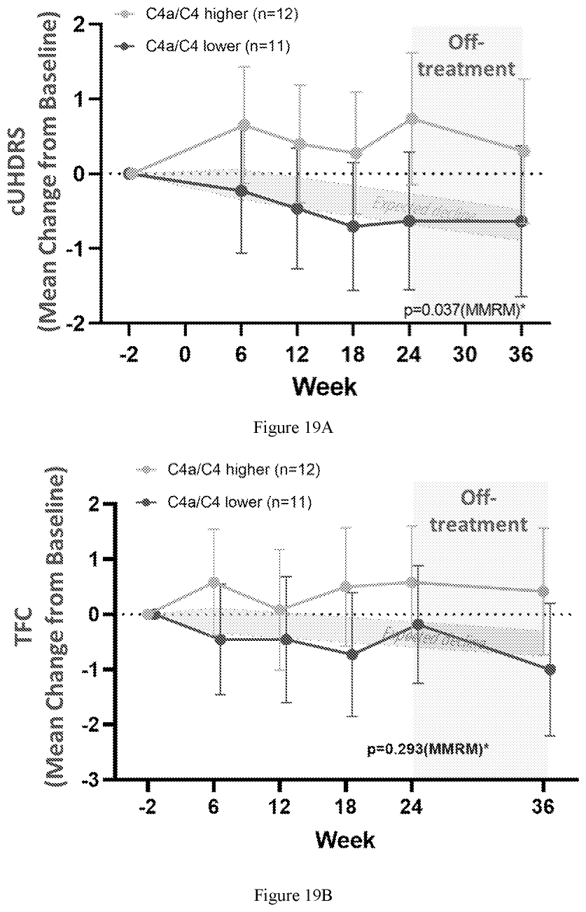

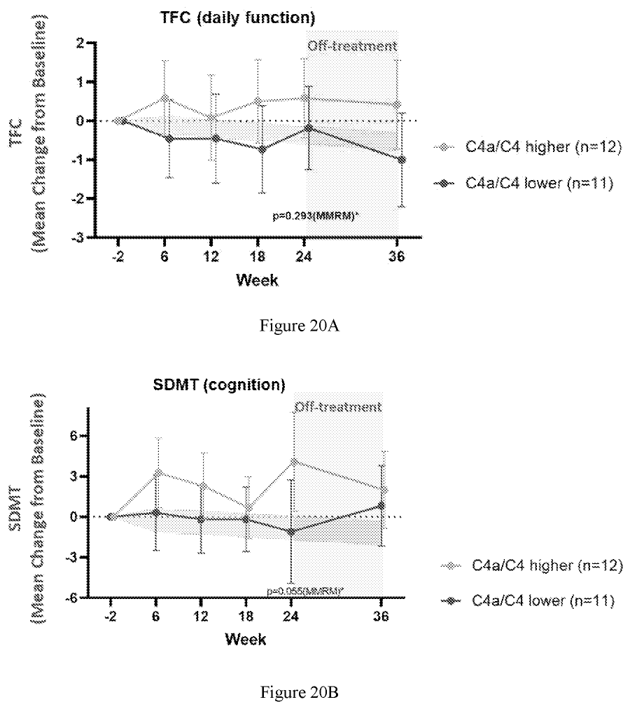

Full-length C1q antibody treatment resulted in early and sustained improvement in patients with excess baseline complement activity. FIGS. 19A-19B show significant improvement during treatment period for subjects with excess versus lower baseline complement activity. FIGS. 20A-20D show that benefit in patients with high complement activity was demonstrated across most cUHDRS constituent domains. FIG. 20A shows total functional capacity (TFC, activities of daily living). FIG. 20B shows Symbol Digit Modality Test (SDMT, Cognition). FIG. 20C shows total motor score (TMS). FIG. 20D shows stroop word reading test (SWR, Cognition).

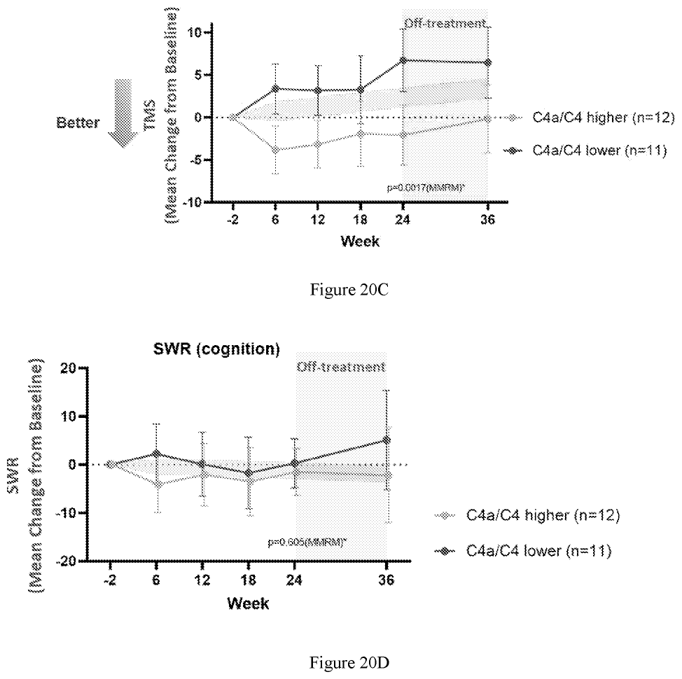

Plasma NfL levels were stabilized throughout study. FIG. 21 shows that plasma NfL levels are consistent with HD natural history at week 36 with 4% change from screening (Rodriguez, et al., Sci Transl Med. 2020 12 16; 12(574)) and (Tabrizi, NEJM 2019, 38:2307). FIG. 14 shows that CSF NfL levels are consistent with HD natural history at week 36 with 16% change from screening. *Results are independent of baseline complement activity.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize or be able to ascertain using no more than routine experimentation, many equivalents to the specific embodiments of the invention described herein. Such equivalents are intended to be encompassed by the following claims.

What is claimed is:

1. A method of treating Huntington's disease in a subject in need thereof, the method comprising: determining that the subject has an elevated level of C4a or an elevated C4a/C4 ratio; and administering to the subject an inhibitor of the classical complement pathway.

2. The method of claim 1, wherein the elevated level of C4a is greater than a C4a level in normal or healthy subjects.

3. The method of claim 1, wherein the elevated level of C4a is greater than a C4a level in normal or healthy subjects of a similar age.

4. The method of any one of claims 1-3, wherein the elevated level of C4a is greater than a reference C4a level.

5. The method of claim 4, wherein the reference C4a level is a value that is equal to or greater than the median of C4a levels in samples derived from Huntington's disease subjects.

6. The method of claim 4, wherein the reference C4a level is a value that is equal to or greater than the median of C4a levels in samples derived from Huntington's disease subjects of a similar age.

7. The method of claim 4, wherein the reference C4a level is a value that is equal to or greater than the 75th percentile of C4a levels in samples derived from normal or healthy subjects.

8. The method of claim 4, wherein the reference C4a level is a value that is equal to or greater than the 75th percentile of C4a levels in samples derived from normal or healthy subjects of a similar age.

9. The method of any one of claims 5-8, wherein the normal or healthy subjects do not have Huntington's disease.

10. The method of any one of claims 3-9, wherein the elevated level of C4a is greater than the reference C4a level by at least 1%, 2%, 3%, 4%, 5%, 6%, 7%, 8%, 9%, 10%, 15%, 20%, 25%, 30%, 35%0, 40%, 45%, 50%, 55%, 60%, 65%, 70%, 75%, 80%, 85%, 90%, 95%, 100%, 200%, 300%, 400%, or 500%.

11. The method of any one of claims 6-9, wherein the elevated level of C4a is greater than the reference C4a level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

12. The method of any one of claims 1-11, wherein the elevated C4a/C4 ratio is greater than a C4a/C4 ratio in normal or healthy subjects.

13. The method of any one of claims 1-11, wherein the elevated C4a/C4 ratio is greater than a C4a/C4 ratio in normal or healthy subjects of a similar age.

14. The method of any one of claims 1-13, wherein the elevated C4a/C4 ratio is greater than a reference C4a/C4 ratio.

15. The method of claim 14, wherein the reference C4a/C4 ratio is a value that is equal to or greater than the median of C4a/C4 ratio in samples derived from Huntington's disease subjects.

16. The method of claim 14, wherein the reference C4a/C4 ratio is a value that is equal to or greater than the median of C4a/C4 ratio in samples derived from Huntington's disease subjects of a similar age.

17. The method of claim 14, wherein the reference C4a/C4 ratio is a value that is equal to or greater than the 75th percentile of C4a/C4 ratios in samples derived from normal or healthy subjects of.

18. The method of claim 14, wherein the reference C4a/C4 ratio is a value that is equal to or greater than the 75th percentile of C4a/C4 ratios in samples derived from normal or healthy subjects of a similar age.

19. The method of any one of claim 15-18, wherein the normal or healthy subjects do not have Huntington's disease.

20. The method of any one of claims 14-19, wherein the elevated C4a/C4 ratio is greater than the reference C4a/C4 ratio by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, or 500%.

21. The method of any one of claims 14-19, wherein the elevated C4a/C4 ratio is greater than the reference C4a/C4 ratio by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, or 400%-500%.

22. The method of any one of claims 1-21, wherein the level of C4a or the C4a/C4 ratio is measured in cerebrospinal fluid (CSF) or plasma.

23. The method of any one of claims 1-22, wherein the subject has an elevated level of Neurofilament light chain (NfL).

24. The method of claim 23, wherein the elevated level of NfL is greater than a NfL level in normal or healthy subjects.

25. The method of claim 23, wherein the elevated level of NfL is greater than a NfL level in normal or healthy subjects of a similar age.

26. The method of any one of claims 23-25, wherein the elevated level of NfL is greater than a reference NfL level.

27. The method of claim 26, wherein the reference NfL level is about 100 pg/ml, 200 pg/ml, 300 pg/ml, 400 pg/ml, 500 pg/ml, 600 pg/ml, 700 pg/ml, 800 pg/ml, 900 pg/ml, 1000 pg/ml, 1100 pg/ml, 1200 pg/ml, 1300 pg/ml, 1400 pg/ml, 1500 pg/ml, 1600 pg/ml, 1700 pg/ml, 1800 pg/ml, 1900 pg/ml, or 2000 pg/ml.

28. The method of any one of claims 24-27, wherein the elevated level of NfL is greater than the NfL level in normal or healthy subjects or the reference NfL level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000%, 4000%, or 5000%.

29. The method of any one of claims 24-27, wherein the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%.

30. The method of any one of claims 23-29, wherein the level of NfL is measured in cerebrospinal fluid (CSF).

31. The method of any one of claims 23-29, wherein the level of NfL is measured in plasma.

32. The method of claim 31, wherein the reference NfL level is about 1 pg/ml, 2 pg/ml, 3 pg/ml, 4 pg/ml, 5 pg/ml, 6 pg/ml, 7 pg/ml, 8 pg/ml, 9 pg/ml, 10 pg/ml, 11 pg/ml, 12 pg/ml, 13 pg/ml, 14 pg/ml, 15 pg/ml, 16 pg/ml, 17 pg/ml, 18 pg/ml, 19 pg/ml, or 20 pg/ml.

33. The method of claim 31 or 32, wherein the elevated level of NfL is greater than the NfL level in normal or healthy subjects or the reference NfL level by at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, 2000%, 3000%, 4000%, or 5000%.

34. The method of claim 31 or 32, wherein the elevated level of NfL is greater than the NfL level in normal or healthy subjects or the reference NfL level by at least 1%-10%, 10%-20%, 20%-30%, 30%-40%, 40%-50%, 50%-60%, 60%-70%, 70%-80%, 80%-90%, 90%-100%, 100%-200%, 200%-300%, 300%-400%, 400%-500%, 500%-600%, 600%-700%, 700%-800%, 800%-900%, 900%-1000%, 1000%-2000%, 2000%-3000%, 3000%-4000%, or 4000-5000%.

35. The method of any one of claims 1-34, wherein the inhibitor of the classical complement pathway is a C1q inhibitor.

36. The method of claim 35, wherein the C1q inhibitor is a small molecule, an antibody, an aptamer, an antisense nucleic acid or a gene editing agent.

37. The method of claim 36, wherein the antibody is an anti-C1q antibody.

38. The method of claim 37, wherein the antibody is administered at a dose of at least 75 mg/kg.

39. The method of claim 38, wherein the antibody is administered at a dose of 75 mg/kg.

40. The method of claim 38, wherein the antibody is administered at a dose of 100 mg/kg.

41. The method of claim 38, wherein the antibody is administered at a dose of 75 mg/kg on day 1 and on day 5 or day 6.

42. The method of claim 41, wherein the antibody is further administered at a dose of 100 mg/kg every two weeks.

43. The method of any one of claims 37-42, wherein the antibody is administered intravenously.

44. The method of any one of claims 37-43, wherein the antibody is administered once a week.

45. The method of any one of claims 37-43, wherein the antibody is administered once every other week.

46. The method of any one of claims 37-43, wherein the antibody is administered once a month.

47. The method of any one of claims 37-43, wherein the antibody is administered once every six weeks.

48. The method of any one of claims 37-43, wherein the antibody is administered once every other month.

49. The method of any one of claims 37-48, wherein the antibody is administered for at least 3 months, 6 months, at least 7 months, at least 8 months, at least 9 months, at least 10 months, at least 11 months, or at least 12 months.

50. The method of claim 49, wherein the antibody is administered for 6 months.

51. The method of any one of claims 37-50, wherein the anti-C1q antibody inhibits the interaction between C1q and an autoantibody or between C1q and C1r, or between C1q and C1s.

52. The method of any one of claims 37-50, wherein the anti-C1q antibody promotes clearance of C1q from circulation or a tissue.

53. The method of any one of claims 37-52, wherein the antibody is a monoclonal antibody, a polyclonal antibody, a recombinant antibody, a humanized antibody, a chimeric antibody, a multispecific antibody, antibody fragments, or an antibody derivative thereof.

54. The method of claim 53, wherein the antibody fragment is a Fab fragment, a Fab′ fragment, a F(ab′)2 fragment, a Fv fragment, a diabody, a single chain antibody molecule, or a single arm antibody molecule.

55. The method of any one of claims 37-54, wherein the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7.

56. The method of any one of claims 37-55, wherein the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11.

57. The method of any one of claims 37-56, wherein the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7.

58. The method of claim 57, wherein the light chain variable domain comprising an amino acid sequence selected from SEQ ID NO: 4 and 35-38.

59. The method of any one of claims 37-58, wherein the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11.

60. The method of claim 59, wherein the heavy chain variable domain comprising an amino acid sequence selected from SEQ ID NO: 8 and 31-34.

61. The method of any one of claims 53-56, wherein the antibody fragment comprises heavy chain Fab fragment of SEQ ID NO: 39 and light chain Fab fragment of SEQ ID NO: 40.

62. The method of any one of claims 1-34, wherein the inhibitor of the classical complement pathway is a C1r inhibitor.

63. The method of claim 62, wherein the C1r inhibitor is a small molecule, an antibody, an aptamer, an antisense nucleic acid or a gene editing agent.

64. The method of claim 63, wherein the antibody is an anti-C1r antibody.

65. The method of claim 64, wherein the anti-C1r antibody inhibits the interaction between C1r and C1q or between C1r and C1s, or wherein the anti-C1r antibody inhibits the catalytic activity of C1r or inhibits the processing of pro-C1r to an active protease.

66. The method of any one of claims 1-34, wherein the inhibitor of the classical complement pathway is a C1s inhibitor.

67. The method of claim 66, wherein the C1s inhibitor is a small molecule, an antibody, an aptamer, an antisense nucleic acid or a gene editing agent.

68. The method of claim 67, wherein the antibody is an anti-C1s antibody.

69. The method of claim of 68, wherein the anti-C1s antibody inhibits the interaction between C1s and C1q or between C1s and C1r or between C1s and C2 or C4, or wherein the anti-C1s antibody inhibits the catalytic activity of C1s or inhibits the processing of pro-C1s to an active protease or binds to an activated form of C1s.

70. The method of claim 68, wherein the antibody is sutimlimab.

71. The method of any one of claims 1-34, wherein the inhibitor of the classical complement pathway is an anti-C1 complex antibody, optionally wherein the anti-C1 complex antibody inhibits C1r or C1s activation or blocks their ability to act on C2 or C4.

72. The method of claim 71, wherein the anti-C1 complex antibody binds to a combinatorial epitope within the C1 complex, wherein said combinatorial epitope comprises amino acids of both C1q and C1s; both C1q and C1r; both C1r and C1s; or each of C1q, C1r, and C1s.

73. The method of any one of claims 1-34, wherein the inhibitor of the classical complement pathway is a C2 inhibitor.

74. The method of claim 73, wherein the C2 inhibitor is a small molecule, an antibody, an aptamer, an antisense nucleic acid or a gene editing agent.

75. The method of any one of claims 1-34, wherein the inhibitor of the classical complement pathway is a C3 inhibitor.

76. The method of claim 75, wherein the C3 inhibitor is a small molecule, an antibody, an aptamer, an antisense nucleic acid or a gene editing agent.

77. The method of claim 76, wherein the C3 inhibitor is APL-9 (Apellis) or AMY-101 (Amyndas).

78. The method of any one of claims 1-34, wherein the inhibitor of the classical complement pathway is a C4 inhibitor.

79. The method of claim 78, wherein the C4 inhibitor is a small molecule, an antibody, an aptamer, an antisense nucleic acid or a gene editing agent.

80. A method of treating Huntington's disease in a subject in need thereof, the method comprising: determining that the subject has an elevated level of C4a or an elevated C4a/C4 ratio; and administering to the subject an antibody having a light variable domain comprising an amino acid sequence of SEQ ID NO: 37 and a heavy chain variable domain comprising the amino acid sequence of SEQ ID NO: 33.

81. The method of claim 80, wherein the antibody is administered intravenously at a dose of at least 75 mg/Kg on day 1 and day 5 or day 6.

82. The method of claim 81, wherein the antibody is further administered intravenously at a dose of 100 mg/Kg every two weeks.

83. The method of any one of claims 80-82, wherein the antibody comprises a heavy chain comprising an amino acid sequence of SEQ ID NO: 14 and a light chain comprising an amino acid sequence of SEQ ID NO: 40.


SEQUENCE LISTING (47 sequences)

SEQ ID NO: 1 - protein, 245 aa, Homo sapiens
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 448 aa, Homo sapiens
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 4 - protein, 107 aa, synthetic construct
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, synthetic construct
RASKSINKYLA

SEQ ID NO: 6 - protein, 7 aa, synthetic construct
SGSTLQS

SEQ ID NO: 7 - protein, 9 aa, synthetic construct
QQHNEYPLT

SEQ ID NO: 8 - protein, 121 aa, synthetic construct
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, synthetic construct
GYHFTSYWMH

SEQ ID NO: 10 - protein, 17 aa, synthetic construct
VIHPNSGSINYNEKFES

SEQ ID NO: 11 - protein, 12 aa, synthetic construct
ERDSTEVLPMDY

SEQ ID NO: 12 - DNA, 321 bp, synthetic construct
  source 1..321: mol_type = other DNA
gatgtccagataacccagtctccatcttatcttgctgcatctcctggagaaaccattactattaattgcagggcaagtaagagcattaacaaatatttagcctggtatcaagagaaacctgggaaaactaataagcttcttatctactctggatccactttgcaatctggaattccatcaaggttcagtggcagtggatctggtacagatttcactctcaccatcagtagcctggagcctgaagattttgcaatgtattactgtcaacaacataatgaatacccgctcacgttcggtgctgggaccaagctggagctgaaa

SEQ ID NO: 13 - DNA, 363 bp, synthetic construct
  source 1..363: mol_type = other DNA
caggtccaactgcagcagcctggggctgagctggtaaagcctggggcttcagtgaagttgtcctgcaagtcttctggctaccatttcaccagctactggatgcactgggtgaagcagaggcctggacaaggccttgagtggattggagtgattcatcctaatagtggtagtattaactacaatgagaagttcgagagcaaggccacactgactgtagacaaatcctccagcacagcctacatgcaactcagcagcctgacatctgaggactcggcggtctattattgtgcaggagagagagattctacggaggttctccctatggactactggggtcaaggaacctcagtcaccgtctcctca

SEQ ID NO: 14 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 12 aa, synthetic construct
SSVSSSYLHWYQ

SEQ ID NO: 16 - protein, 10 aa, synthetic construct
STSNLASGVP

SEQ ID NO: 17 - protein, 10 aa, synthetic construct
HQYYRLPPIT

SEQ ID NO: 18 - protein, 12 aa, synthetic construct
GFTFSNYAMSWV

SEQ ID NO: 19 - protein, 10 aa, synthetic construct
ISSGGSHTYY

SEQ ID NO: 20 - protein, 11 aa, synthetic construct
ARLFTGYAMDY

SEQ ID NO: 21 - protein, 109 aa, synthetic construct
DIVMTQTTAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 22 - protein, 115 aa, synthetic construct
QVKLEESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVT

SEQ ID NO: 23 - protein, 109 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIK

SEQ ID NO: 24 - protein, 118 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSS

SEQ ID NO: 25 - protein, 216 aa, synthetic construct
QIVLTQSPATLSLSPGERATMSCTASSSVSSSYLHWYQQKPGKAPKLWIYSTSNLASGVPSRFSGSGSGTDYTLTISSLQPEDFATYYCHQYYRLPPITFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 445 aa, synthetic construct
EVQLVESGGGLVKPGGSLRLSCAASGFTFSNYAMSWVRQAPGKGLEWVATISSGGSHTYYLDSVKGRFTISRDNSKNTLYLQMNSLRAEDTALYYCARLFTGYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 12 aa, synthetic construct
TASSSVSSSYLH

SEQ ID NO: 28 - protein, 7 aa, synthetic construct
STSNLAS

SEQ ID NO: 29 - protein, 5 aa, synthetic construct
NYAMS

SEQ ID NO: 30 - protein, 17 aa, synthetic construct
TISSGGSHTYYLDSVKG

SEQ ID NO: 31 - protein, 121 aa, synthetic construct [sequence omitted]

SEQ ID NO: 32 - protein, 121 aa, synthetic construct [sequence omitted]

SEQ ID NO: 33 - protein, 121 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 34 - protein, 121 aa, synthetic construct [sequence omitted]

SEQ ID NO: 35 - protein, 107 aa, synthetic construct [sequence omitted]

SEQ ID NO: 36 - protein, 107 aa, synthetic construct [sequence omitted]

SEQ ID NO: 37 - protein, 107 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, synthetic construct [sequence omitted]

SEQ ID NO: 39 - protein, 229 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 253 aa, Homo sapiens
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 42 - protein, 245 aa, Homo sapiens
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 43 - protein, 9 aa, synthetic construct
LFTGYAMDY

SEQ ID NO: 44 - protein, 109 aa, synthetic construct
QIVLTQSPAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 45 - protein, 118 aa, synthetic construct
EVMLVESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVTVSS

SEQ ID NO: 46 - skipped

SEQ ID NO: 47 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK